(19) United States
(12) Patent Application Publication
(10) Pub. No.: US 2015/0306042 A1
(43) Pub. Date: Oct. 29, 2015

(54) BODY SURFACE TREATMENT COMPOSITION AND METHOD

(71) Applicant: ARMS PHARMACEUTICAL LLC, Cleveland, OH (US)

(72) Inventors: Afif Mahmoud Ghannoum, Shaker Heights, OH (US); Brian Vincent Sokol, Vermilion, OH (US)

(21) Appl. No.: 14/758,902

(22) PCT Filed: Jan. 3, 2014
(86) PCT No.: PCT/US2014/010174

§ 371 Date: Jul. 1, 2015

Related U.S. Application Data
(63) Continuation-in-part of application No. 14/063,974, filed on Oct. 25, 2013.
Application No. 14/063,974 is a continuation-in-part of application No. 13/734,363, filed on Jan. 4, 2013, now abandoned.
(60) Provisional application No. 61/749,195, filed on Jan. 4, 2013.
(60) Provisional application No. 61/829,608, filed on May 31, 2013.

(51) Int. Cl. A61K 9/70; A61K 9/08; A61K 9/00; A61K 31/4425; A61K 31/35

(52) U.S. Cl. CPC A61K 9/7015; A61K 31/4425; A61K 31/35; A61K 9/0014; A61K 9/08

(57) ABSTRACT

A method for treating a body surface with an active agent, includes: administering a composition in a therapeutically effective amount to a body surface, the composition forming a barrier coating on the body surface that is active to sustain contact with the body surface for a duration of at least about one hour. The composition meets the following requirements: about 0.0001%<C<about 0.4%, about 0.07%<H<about 70%, optionally an antimicrobial A, and a therapeutically effective amount of X; or 0%<C<about 0.4%, about 55%<H<about 70%, optionally an antimicrobial A, and a therapeutically effective amount of X. All percentages are by weight of the total composition and C is a carbohydrate gum; H is a humectant; and A is the active agent, the active agent being active to produce a therapeutic effect on the body.

### CROSS-REFERENCE TO RELATED APPLICATIONS

This application claims the benefit of priority to U.S. 61/749,195, filed on Jan. 4, 2013, titled, “Barrier-Forming Composition with Active Agents and Method.” The present application also claims the benefit of priority and is a continuation-in-part of U.S. application Ser. No. 14/063,974, filed on Oct. 25, 2013, titled “Low-Tack Surface Anti-Microbial Coating Composition and Method of Application.” The present application also claims the benefit of priority and is a continuation-in-part of U.S. application Ser. No. 13/734,363, filed Jan. 4, 2013, titled “Long-Lasting Surface Anti-Microbial and Method of Application.” The present application also claims the benefit of priority to U.S. provisional application No. 61/829,608, filed on May 31, 2013, titled “Method for Reducing Microbial Load for Treatment of Disease Caused or Aggravated by Microorganisms or Relieving Symptoms Thereof.” All of these prior applications are incorporated herein by reference for all purposes.

### FIELD

This disclosure relates to a skin antimicrobial composition and methods for treating human body surfaces with topical active agents.

### BACKGROUND

Numerous topical compositions for treating human body surfaces such as skin, lesions, or mucosal surfaces are available. Such compositions include active agents for treating various conditions and maladies. Topical compositions, however, are often easily washed away, or otherwise have limited effectiveness because they are not kept in contact with the body surface for a duration long enough to maximize their effectiveness. Many topical compositions are intermittent, transitory options that do not provide sustained protection.

### SUMMARY

A method for treating a body surface with an active agent, includes: administering a composition in a therapeutically effective amount to a body surface, the composition forming a barrier coating on the body surface that is active to sustain contact with the body surface for a duration of at least about one hour. The composition meets the following requirements: about 0.0001%≦C≦about 0.4%, about 0.07%≦H≦about 70%, optionally an antimicrobial A, and a therapeutically effective amount of X; or 0%≦C≦about 0.4%, about 55%≦H≦about 70%, optionally an antimicrobial A, and a therapeutically effective amount of X. All percentages are by weight of the total composition and C is a carbohydrate gum; H is a humectant; and A is the active agent, the active agent being active to produce a therapeutic effect on the body.

A method for killing microorganisms on a mammal skin surface includes: applying a composition that comprises an antimicrobial agent onto the skin surface; in the coating, killing or neutralizing microorganisms encountered on the surface and encountered from the environment after the applying step is performed; forming a coating layer on the skin surface. The composition includes a humectant and an antimicrobial agent. The antimicrobial comprises a monoquaternary ammonium compound or pharmaceutically acceptable salt thereof. The coating layer includes antimicrobial cidal or static activity for at least about one hour.

A composition for body surface treatment includes: about 0.0001%≦C≦about 0.4%; about 0.07%≦H≦about 70%, optionally an antimicrobial A, and a therapeutically effective amount of X; or 0%≦C≦about 0.4%, about 55%≦H≦about 70%, optionally an antimicrobial A, and a therapeutically effective amount of X. All percentages are by weight of the total composition and C is a carbohydrate gum, H is a humectant, A is an antimicrobial, and X is an active agent. The active agent is selected from the group consisting of one or more of: antacids, probiotics, vitamins, nutraceuticals, silver, anti-oxidants, cold and flu symptom medicaments, anti-prions, immunostimulators, anti-diarrheals, anti-nausea, topical anesthetics, topical analgesics, anti-pruritics (anti-itch), anti-allergen medicaments, topical steroids, decongestants, cough suppressants, burn or sunburn treatments, expectorants, anti-histamines, topical NSAIDs, topical salicylates, hair-regrowth agents, erectile dysfunction treatment agents, topical antibiotics, topical wound care agents, antioxidant agents, hemorrhoid treatment agents, topical antifungal agents, anti-acne agents, scar treatment agents, psoriasis treatment agents, dry scalp treatment agents, anti-dandruff agents, lice killing agents, hair care agents, body odor amelioration agents, antiperspirants, and foot odor amelioration agents.

The articles “a” and “the,” as used herein, mean “one or more” unless the context clearly indicates to the contrary.

The terms “item” and “apparatus” are used synonymously herein.

The term “therapeutic,” as used herein, is meant to also apply to preventative treatment.

The term “or,” as used herein, is not an exclusive or, unless the context clearly indicates to the contrary.

The use of the term “individual” or “mammal” herein, means a human or animal commonly defined as a mammal.

The term “lesion” is used herein interchangeably with the term “disruption.”

The use of the term “block” or “blocking” herein, includes blocking passage by trapping.

### BRIEF DESCRIPTION OF THE DRAWINGS

FIG. 1A is an illustrated flow chart of a treated and untreated inanimate surface and the results of an encounter with microorganisms.

FIG. 1B is a depiction of a proposed mechanism of antimicrobial activity in an embodiment of the coating composition.

FIG. 2 is a schematic showing the formation of a barrier on a mucosal surface, as described in Example 2.

FIG. 3 is a schema showing the method of evaluation of microbial growth in the upper and lower chambers of an EHOM assay, as described in Examples 27-28.

FIG. 4 show photographs of agar media plates showing microbial growth in the upper and lower chambers of an EHOM assay, as described in Examples 27-28.

FIG. 5 shows photographs of magnified cross-sections of the coating-composition-treated and untreated engineered human oral mucosa (EHOM) of Examples 31-32.

FIG. 6 shows photographs of microbial growth on untreated EHOM or EHOM treated with an example coating composition, followed by infection with C. albicans, as described in Examples 33-40.

FIG. 7 shows photographs of microbial growth on untreated EHOM or EHOM treated with formulations followed by infection with S. mutans, as described in Examples 33-40.

FIG. 8 shows photographs of microbial growth from “flow-through” media (collected from the lower chamber) of EHOM treated with an example coating composition, as described in Example 33-40.

FIG. 9 presents graphs showing LDH release by EHOM treated with saline (control) or example coating compositions, followed by infection with (A) C. albicans or (B) S. mutans, as described in Examples 40-47.

FIG. 10 is a graph showing post-antimicrobial effect of coating compositions against bacteria and fungi, as described in Examples 63-69.

FIG. 11 shows scanning electron micrographs of S. sanguis, C. albicans, and S. mutans, untreated or treated with coating composition, as described in Examples 71-76.

FIG. 12 presents graphs depicting activity of an example coating composition against biofilms formed by bacteria and fungi, as described in Examples 77-79.

FIG. 13 is a graph showing activity of an example coating composition on microbial biofilms after a 1-min exposure, as described in Examples 80-81.

FIG. 14 presents fluorescent microscopy photographs showing the effect of an example coating composition on cytopathic effects (CPE) of influenza (H1N1)-infected MDCK cells, as described in Examples 85-86.

FIG. 15 presents fluorescent microscopy photographs showing the effect of an example coating composition on against H1N1 virus, as described in Examples 85-86.

FIG. 16 is a graph showing levels of influenza virus in infected coating composition treated and -untreated cells, as determined by quantitative PCR, as described in Examples 87-88.

FIG. 17 is a graph showing direct antiviral activity of example coating compositions prepared with or without preservatives and antimicrobial agent (CPC) against influenza virus, determined using quantitative PCR, as described in Examples 89-91.

FIG. 18 shows the activity of an example coating composition against H1N1 virus over a 6 hour time period. Panel (A) is a graph showing a percent inhibition in viral growth compared to an untreated control. Panels (B) and (C) are micrographs of (B) untreated and (C) coating composition treated cells.

FIG. 19 is a graph showing the activity of formulations against HIV, as described in Examples 94-96.

FIG. 20 is a Western blot showing activity of Example 8 against Epstein-Barr Virus (EBV), as described in Example 97.

FIG. 21 are photographs demonstrating the ability of an example coating composition to coat the oral mucosal surface.

FIG. 22 are photographs showing time-lapse microscopy of bacterial growth after a 1 minute exposure to an example coating composition, as described in Examples 162-163. Images represent bacterial growth after 20 min, 120 min, or 360 min post-exposure.

FIG. 23 is a graph showing the effect of a single dose of an example coating composition on oral microbial burden of a healthy individual, as described in Example 164-166. (A)—Microbial load in CFUs, (B) reduction in microbial load (%) compared to baseline.

FIG. 24 is a graph showing the effect of an example coating composition on levels of oral microbes over a 5-day period in three healthy adults, as described in Examples 167-169.

FIG. 25 is a graph showing the effect of an example coating composition on microbial burden of the oral cavity after 5-day usage in 31 healthy subjects, as described in Examples 170-198.

FIG. 26 is a graph showing the microbial load in oral samples obtained from three representative study participants, as described in Examples 170-198.

FIG. 27 shows a schema describing the in vitro filter insert-based model to evaluate penetration of microbes across the barrier formed by example coating compositions, as described in Examples 199-205.

FIG. 28 is a set of photographs showing growth of MRSA biofilms on the surface of silicone elastomer discs treated with PBS (control, A, C, E) and Example 7 coating composition (B, D, F), as described in Examples 219-224.

FIG. 29 is a set of photographs showing cell monolayers treated with an embodiment of the coating composition, Example 252, for varying time periods (a), (b), and (c), and a control Example 253 (d).

FIG. 30 is a set of immunofluorescence photographs showing cell monolayers treated with an embodiment of the coating composition, Example 252, for varying time periods (a), (b), and (c), and a control Example 253 (d).

FIG. 31 is a graph showing a test composition and a comparison after a water-wash that corresponds to Example 255.

FIG. 32 shows photographs depicting a gum-containing embodiment in comparison to a no-gum embodiment demonstrating tackiness, viscosity, and thickness differences.

FIG. 33 shows photographs depicting a gum-containing embodiment in comparison to a no-gum embodiment demonstrating viscosity, thickness, and surface coverage differences.

FIG. 34 shows photographs of agar plates imprinted with infected and treated pig skin samples and corresponds to Example 261.

FIG. 35 shows photographs of agar plates imprinted with infected and treated pig skin samples and corresponds to Example 262.

### DETAILED DESCRIPTION

In an embodiment, a stable solution composition with various active agents is disclosed for topical treatment of mammal body surfaces. In an embodiment, the composition is non-toxic to mammals and safe (i.e. does not cause damage to the mucosa or other body surfaces) in a therapeutically effective amount.

In an embodiment, a topical antimicrobial composition functions to inhibit microbial growth through static or cidal activity for an extended period of time. In an embodiment, the composition is long-lasting, powerful, and kills a broad-spectrum of microorganisms (e.g. fungi, bacteria, and viruses), such as an antiseptic. The composition acts to neutralize and/or kill microorganisms previously deposited on the treated surface and/or microorganisms that subsequently come into contact with an exposed (top) surface of the applied antimicrobial coating. In an embodiment, the composition is a barrier-forming coating that is effective to trap and kill or neutralize microorganisms already present on the treated surface and/or to trap and kill or neutralize microorganisms that are subsequently deposited on top of the barrier, i.e., the exposed surface of the barrier coating, after the administering of the barrier forming composition is performed.

In an embodiment the composition is a stable, barrier-forming composition that enhances the duration and efficacy of active agents on body surfaces. In an embodiment, the barrier-forming composition is non-toxic to mammals and safe (i.e. does not cause damage to the mucosa or other body surfaces) in a therapeutically effective amount. The barrier-forming composition forms a barrier coating that sustains active agents in contact against the body surface. Various active agents may be included in embodiments of the composition.

In an embodiment the composition is a topical antimicrobial coating for treatment of mammal skin. In an embodiment, the topical antimicrobial for treatment of mammal skin is a low tack variation of the composition. Other embodiments of the topical antimicrobial for skin may also include gum and/or higher concentrations of humectant as stated below.

In a low tack embodiment, the coating composition meets the following requirements:

about 0.07%≦H≦about 10%; and

0.0005%<A

wherein H is a humectant and A is an antimicrobial.

All percentages are by weight of the total composition.

In an embodiment, the concentration of the humectant may range about 3% to about 8%, 0.35% to less than 1%, or about 0.1% to less than 0.5%. In another embodiment, the humectant of the coating composition meets the following requirements: about 0.07%≦H≦1%. This low-humectant embodiment reduces the stickiness or adhesiveness of the composition to provide a better tactile sensation to the inanimate surface. The antimicrobial may be present in a range of 0.0005% to 5% by weight. Further ranges of the antimicrobial are also stated below.

In the low tack embodiment, the coating composition is essentially free of carbohydrate gum, such as, for example, including less than 0.00009% carbohydrate gum, no detectable carbohydrate gum, or no carbohydrate gum. The term essentially free also includes completely free.

The composition may comprise about 50% to about 98% by weight water, such as, for example, about 75% to about 97% water, or about 85% to about 95% water.

In an embodiment, the composition consists essentially of only the humectant and the antimicrobial, such as, additionally including only preservatives, scenting agents, or other agents, that do not affect the trapping or antimicrobial activity of the composition. “Consists essentially of” or “consisting essentially of” as used herein has the meaning that is typically applied, that is, it means, the specified materials and those that do not materially affect the basic and novel characteristic(s) of the composition. In some embodiments, this may include less than 1%, less than 0.01%, or less than 0.0001% by weight of non-specified materials. In an embodiment, the low tack composition may include any of the additional active agents listed herein.

In such an embodiment, when utilized as a skin topical antimicrobial, the antimicrobial may be is a monoquaternary ammonium compound or a pharmaceutically acceptable salt thereof, such as, for example, QAC, cetrimide, benzalkonium chloride, cetalkonium chloride, cetylpyridinium chloride, myristalkonium chloride, and Polycide. In an embodiment, the antimicrobial component of the composition may consist essentially of the monoquaternary ammonium compound or a pharmaceutically acceptable salt thereof, Cetylpyridinium chloride has shown excellent effectiveness with only a small amount of humectant and preservatives, scenting agents, and/or other agents, that do not affect the trapping or antimicrobial activity of the composition. Such a composition has several safety and environmental advantageous over other skin sanitizing topical compositions. See Example 256. In an embodiment the antimicrobial component, that is the only agents acting as antimicrobials, consists essentially of cetylpyridinium chloride.

In an embodiment, the composition comprises a carbohydrate gum (C), a humectant (H), optionally an antimicrobial agent (A), and an active agent (X), and the barrier-forming composition meets the following requirements:

about 0.0001%≦C≦about 0.4%;

about 0.07%≦H≦about 70%;

optionally 0.0005%<A; and

a therapeutically effective amount of X;

or

0%≦C≦about 0.4%;

about 55%≦H≦about 70%;

optionally 0.0005%<A; and

a therapeutically effective amount of X.

All percentages are by weight of the total composition. After effective application, the barrier layer that includes the optional antimicrobial has antimicrobial cidal or static activity. The therapeutically effective amount of X will vary according to the active agent used, but will generally be no more than the effective amount of the active agent in its typical dosage amount as taught in the prior art outside of a combination with the barrier-forming composition. For example, the active agent may be present in an amount of at least about 0.0005%, such as about 0.001% to about 0.01%, about 0.1% to about 1%, or about 2% to about 50%. In an embodiment, the amount of X is less than a known dosage of the agent in the prior art, such as 10% to 95% less than a known prior art minimum effective dosage, or 25% to 75% less than a known prior art minimum effective dosage, or 50% or less than a known prior art minimum effective dosage.

In another embodiment the barrier-forming composition meets the following requirements:

about 0.01%≦C≦about 0.4%;

about 4.5%≦H≦about 65%;

optionally 0.0005%<A; and

a therapeutically effective amount of X;

or

0%≦C≦about 0.4%;

about 55%≦H≦about 65%;

optionally 0.0005%<A; and

a therapeutically effective amount of X.

wherein C is a carbohydrate gum; H is a humectant; A is an antimicrobial; and X is an active agent, the active agent being active to produce a therapeutic effect on the body.

In an embodiment of the barrier-forming composition with one or more active agents the composition meets the following requirements:

about 0.0001%≦C≦about 0.4%;
about 0.07%≦H≦about 70%; and

a therapeutically effective amount of X;

or

0%≦C≦about 0.4%;

about 55%≦H≦about 70%; and

a therapeutically effective amount of X;

wherein all percentages are by weight of the total composition;

wherein C is a carbohydrate gum; H is a humectant; and X is the active agent, the active agent being active to produce a therapeutic effect on the body.

In another embodiment, the humectant of the composition meets the following requirements: about 0.07%<H<1%. This low-humectant embodiment reduces the stickiness of the composition.

In an embodiment, the composition includes glycerin or one or more similar humectant substances. In an embodiment, the concentration of the humectant may range from about 0.07% to about 10% of the entire composition (by weight), such as about 3% to about 8%, 0.35% to less than 1%, or about 0.1% to less than 0.5%. In another embodiment, the humectant may range from about 2% to about 70% weight percent of the entire composition, such as, for example, about 4.5% to about 65%, about 7% to about 35%, or about 15% to about 45%. Humectants similar to glycerin may be classified generally as polyols. The humectants may be, for example, glycerin, sorbitol, xylitol, propylene glycol, polyethylene glycol, and mixtures thereof. In an embodiment, glycerin may be used at high concentrations such as about 55% to about 65% in the absence of a gum.

In an embodiment, the composition also includes a gum. The gum may be, for example, a polysaccharide, xanthan gum, gum Arabic, or guar gum. Such gums may be generally classified as carbohydrate gums that have an overall negative charge. In another embodiment, the gum may be, for example, xanthan gum, guar gum, gum Arabic, tragacanth, gum karaya, locust bean gum, carob gum, and pectin. These gums may also be generally classified as carbohydrate gums that have an overall negative charge. In an embodiment, the gum may be present in a weight percentage of the total composition ranging from about 0.0001% to about 0.4%, such as about 0.0005 to about 0.25%. In another embodiment, the gum may be present in a weight percentage of the total composition ranging from about 0.01% to about 0.4%, such as for example, about 0.25% to about 0.35%, about 0.05% to about 0.25%, or about 0.4%.

In an embodiment, the composition comprises a humectant, an active agent, and optionally a gum, wherein the gum, if present, is present in an amount of about 0.0001% to about 0.4% by weight of the total composition.

In an embodiment, an antimicrobial agent is present in the composition. For example, the composition may include one or more anti-viral agents, or antifungals or antibacterials or a combination thereof. In addition, the effect of such antimicrobials includes static and/or cidal activity.

The antimicrobial agent may include, but is not limited to cationic antimicrobial agents and pharmaceutically acceptable salts thereof, including, for example, monoquaternary ammonium compounds (QAC, cetrimide, benzalkonium chloride, cetalkonium chloride, cetylpyridinium chloride, myristalkonium chloride, Polycide), biquaternaries and bis-biguanides (chlorhexidine, Barquat, hibitane), and biguanides, polymeric biguanides, polyhexamethylene biguanides, Vantocil, Cosmocil, diamidines, halogen-releasing agents including chlorine- and iodine-based compounds, silver and antimicrobial compounds of silver, peracetic acid (PAA), silver sulfadiazine, phenols, bisphenols, hydrogen peroxide, hexachloroprene, halophenols, including but not limited to chloroxylenol (4-chloro-3,5-dimethylphenol; p-chloro-m-xylenol).

In addition, the antimicrobial may also be or include: antibacterial agents, both cidal and static, and different classes, for example tetracycline, chloramphenicol, fusidic acid, fluoroquinolone, macrolide antibacterial agents, oxazolidinones, quinolone- and naphthyridone-carboxylic acid, citral, trimethoprim and sulfamethoxazole (singly and combined), aminoglycoside, polymyxin, penicillins and their derivatives. In addition, the antimicrobial may also include, for example: antifungal agents in the following classes: azoles, polyenes, echinocandins, and pyrimidines. Combinations of the any of the foregoing antimicrobial agents are also contemplated. Many of the foregoing are cationic species or their pharmaceutically acceptable salts, and in an embodiment, cationic antimicrobials are utilized in the composition. In an embodiment the composition is exclusive agents that release gas fumes, such as, for example, chlorine dioxide, or chlorine dioxide producing reactants.

In an embodiment, the composition does not induce mutations or the development of resistance by microbes. This is because of the mechanism of action against the microorganisms by the barrier and the selected antimicrobial.

The optional antimicrobial may be present, for example, in an amount ranging from about 0.0005% to 5% by weight of the total composition, such as, for example, about 0.0025% to about 1%, about 0.005 to about 0.006%, or about 0.0006% to about 0.003%. In another embodiment, the antimicrobial may be present, for example, in an amount ranging from about 0.05% to about 0.1% by weight of the total composition, such as, for example, about 0.05% to about 0.06% or about 0.06% to about 0.1%. In an embodiment, the antimicrobial is about 5% or less, or about 3% or less, or about 1.5% or less, such as when the antimicrobial used does not cause solubility problems at higher concentrations.

In embodiments, the compositions may further include other components, such as, for example, copovidone and other lubricating agents, parabens such as methyl paraben or propylparaben, scenting agents, preservatives, suh as sodium benzoate, buffering agents, such as monosodium and disodium phosphate, sweeteners, hydrogenated castor oil with ethylene oxide, and carboxymethylcellulose. These components may, for example, be included in amounts ranging from about 0.01% to about 5% by weight of the total composition, such as, for example, about 0.1% to about 2%. In another embodiment, the components are included, for example, in amounts of about 0.0001% to about 0.05%. Buffering agents (such as monosodium or disodium phosphate) may also be used.

Purified water and/or alcohol may be used as the diluent component of the composition. In an embodiment, the composition is a free-flowing liquid suitable for spraying. This is in contrast to a paste or toothpaste composition, which is typically not free-flowing and not suitable for spraying. In addition, in an embodiment, the composition is free of abrasives that are commonly used in toothpaste compositions.

In an embodiment, substantially free-flowing and substantially free of clumps is judged by passing the composition through a 140 U.S. mesh (0.10 mm pore size), and 95 to 100% of the composition (by weight), such as 96% to 99.9% passes through, after 30 seconds. In this embodiment the composition is not a gel.

In an embodiment, the composition consists essentially of only the gum, the humectant, and the active agent, such as including only preservatives or scenting agents that do not affect the barrier or active agent's activity. In an embodiment, the composition is exclusive of agents for acting against the teeth and/or gums, including, for example, abrasives (such as those used in toothpastes) teeth whitening or desensitizing agents. In an embodiment, the composition is exclusive of cellooligosaccharides. In an embodiment, the antimicrobial agent is exclusive of lipids such as fatty acid ethers or esters of polyhydric alcohols or alkoxylated derivatives thereof. In an embodiment, the composition is exclusive of one or more of time-release agents, allergy-relief compounds, azelastine, silicon based oils, essential oils, polyvinyl pyrrolidone, polyvinyl alcohol, and potassium nitrate. In an embodiment, the composition is free of volatile organic compounds, including for example, volatile alcohols. In an embodiment, the composition is free of all alcohols. In an embodiment, the composition is free of surfactant or foaming agent. For the avoidance of doubt, none of the above should be construed to mean that all embodiments are exclusive of these compounds.

In an embodiment, a method for making the composition includes mixing and heating the components, including carbohydrate gum, humectant, and optional antimicrobial agent. In an embodiment, heating is replaced with extended mixing times. Other components, including the active agents, may also be mixed in a single or multiple mixing steps. All components of the composition may be mixed at one time to produce a composition with a stable shelf life. This is in contrast to compositions that have active components that must be added separately a short time prior to use. Thus, in an embodiment, the composition is a stable one-part composition that does not require mixing with a second composition to activate it for use.

In an embodiment, the composition is liquid and is non-foaming. In an embodiment, the composition is a solution and not an emulsion or micro-emulsion.

As mentioned above, in an embodiment, the composition is suitable for spraying, and thus also has a viscosity that is suitable for spraying. In an embodiment, the composition has a viscosity of less than 500 cps such as, for example, about 490 cps to about 10 cps, or about 400 cps to about 15 cps. In another embodiment, the composition has a viscosity of about 16 to about 20 cps, such as, for example, about 17 to about 19 cps.

Post antimicrobial effect (PAE) is defined as suppression of microbial growth that persists after limited exposure to an antimicrobial agent. Having a longer PAE is considered advantageous for antimicrobial agents as it allows for persistent inhibition of microbial growth, and may affect dosing regimens as agents with long PAEs may need less frequent administration than those with short PAEs. In embodiments that include an antimicrobial, the composition may have a PAE that persists for about 6 hours or more, such as about 6 hours to about 16 hours, or about 16 hours to about 24 hours.

In an embodiment, the composition is non-toxic to humans, wherein at least a portion of the composition may be ingested and is safe for human consumption. For example, at least a therapeutically effective amount of the composition is safe for human consumption.

Without being bound by theory, the composition is not hydrophilic which allows the composition to have a greater affinity to adhere to and cover certain surfaces. Furthermore, in an embodiment, the antimicrobial being embedded in the non-hydrophilic composition will allow for sustained antimicrobial activity on treated surfaces. In an embodiment the composition is amphiphilic or has amphiphilic components.

One measure of hydrophilicity is the Rf (relative front) value, determined by chromatography in water. In an embodiment, the composition has an Rf value in water of 0 to about 0.25, such as about 0.0001 to about 0.15, or about 0.03 to about 0.1.

In an embodiment, the composition has a pH of about 4 to about 8, such as about 5 to about 7, or about 6 to about 7.5. In another embodiment the composition has a pH of greater than 5.5 to about 8, wherein antimicrobials such as cetylpyridinium chloride are most effective.

In an embodiment the coating composition is non-flammable.

In general, the dual-action mechanism of providing a barrier from microorganisms and an antimicrobial agent provides a long-lasting effect, characterized by both in vitro, simulated in vivo, and in vivo examples below. In in vivo examples, the barrier-forming composition was shown to have antimicrobial effect (cidal or static) for at least 6 hours. The barrier property itself was tested in simulated in vivo tests (on artificial human mucosa EHOMs), which indicated the barrier itself had a significantly extended duration past 6 hours, such as greater than about 8 hours, about 6 to about 16 hours, and about 24 hours, or more. In addition, in vitro tests indicate the antimicrobial effect had a significantly extended duration past about 2 hours, past about 6 hours, and depending on the microorganism tested, greater than about 8 hours, about 6 to about 16 hours, and about 24 hours, or more.

In an embodiment, active agents that may be used in the barrier forming composition are those that have a delayed- or sustained-release property, or otherwise have improved effectiveness the longer they are present on the body surface. Example active agents include antacids, probiotics, vitamins, nutraceuticals, silver, anti-oxidants, cold and flu symptom medicaments, anti-prions, immunostimulators, or combinations of the above. Additional examples, of active agents include, anti-diarrheals, anti-nausea, topical anesthetics, topical analgesics, anti-pruritics (anti-itch), anti-allergen medicaments, topical steroids, decongestants, cough suppressants, burn or sunburn treatments, expectorants, anti-histamines, topical NSAIDs, topical salicylates, hair-regrowth agents, erectile dysfunction treatment agents, topical antibiotics, topical wound care agents, antioxidant agents, hemorrhoid treatment agents, topical antifungal agents, anti-acne agents, scar treatment agents, psoriasis treatment agents, dry scalp treatment agents, anti-dandruff agents, lice killing agents, hair care agents, body odor amelioration agents, antiperspirants, foot odor amelioration agents.

Unless otherwise listed separately below in the more specific agent discussion, the effective amount of the active agent is about the same amount used in conventional medicaments, or less. It is expected that a lower amount of the active agent than what is used conventionally would be required for similar effectiveness due to the barrier-retention property of the composition.

In general, active agents can be included in the composition so long as they are soluble in an effective amount or are at least miscible in an effective amount with the composition, and do not destroy the barrier-forming property of the composition.

In a general embodiment of the barrier-forming composition with one or more active agents, the agents are active for topical treatment of mammal skin. In another general embodiment of the barrier-forming composition with one or more active agents, the agents are active for internal and/or mucosal treatment of mammals.

In an embodiment with a topical anesthetic active agent, the barrier-forming composition is used for numbing pain. The composition may be applied, for example, to the oral or pharyngeal mucosa for example for treating sore throat pain. In another embodiment, the composition may be applied to the skin for example to treat skin or underlying muscle pain. The composition can be sprayed, rubbed, wiped, rolled or otherwise dispersed onto the skin or brought into the oral cavity and swallowed or expectorated. Example topical anesthetics include:

benzocaine, butamben, dibucaine, oxybuprocaine, pramoxine, proparacaine, proxymetacaine, andtetracaine, phenolic compounds, trolamine salicylate or methyl salicylate lidocaine and tetracain.

In an embodiment with an analgesic active agent, the barrier-forming composition is used for reducing pain. The composition may be applied, for example, to the oral or pharyngeal mucosa for example for treating sore throat pain, or for absorption into the blood stream. In another embodiment, the composition may be applied to the skin for example to treat the skin or underlying muscle pain. The composition can be sprayed, rubbed, wiped, rolled or otherwise dispersed onto the skin or mucosa, or brought into the oral cavity and swallowed or expectorated. Example analgesics include: propionic acid derivatives, naproxen, ibuprofen, acetic acid derivatives, indomethacin, etodolac, enolic acid derivatives, fenamic acid derivatives, Cox-2 derivatives, acetaminophen, sulphonanilides, diclofenac, capsaicin, NSAIDs, ibuprofen, trolamine salicylate or methyl salicylate, Menthacin and Zostrix.

In an embodiment with an anti-pruritic active agent, the barrier-forming composition is used for reducing itching on the skin or mucosa. The composition may be applied, for example, to the oral, pharyngeal, vaginal, or anal mucosa. In another embodiment, the composition may be applied to the skin for example to treat itchy skin. The composition can be sprayed, rubbed, wiped, rolled or otherwise dispersed onto the skin or mucosa. Example anti-pruritic agents include, antihistamines such as diphenhydramine, corticosteroids such as hydrocortisone, local anesthetics, such as benzocaine, and counterirritants, such as mint oil, menthol, or camphor.

In an embodiment with an anti-allergen active agent, the barrier-forming composition is used for reducing itching on the skin or mucosa. The composition may be applied, for example, to the oral, pharyngeal, vaginal, or anal mucosa to deliver a dosage of the active agent to the be absorbed in the body's blood stream. In another embodiment, the composition may be applied to the skin for example to treat itchy or irritated skin due to an allergic reaction. The composition can be sprayed, rubbed, wiped, rolled or otherwise dispersed onto the skin or mucosa. Example anti-allergenic agents include, antihistamines such as diphenhydramine, cetirizine, fexofenadine, and eucalyptol.

In an embodiment with a steroid active agent, the barrier-forming composition is used for reducing inflammation on the skin or mucosa. The composition may be applied, for example, to the oral, pharyngeal, vaginal, or anal mucosa to deliver a dosage of the active agent to the be absorbed in the body's blood stream. In another embodiment, the composition may be applied to the skin for example to treat inflamed, itchy, or irritated skin or conditions such as atopic dermatitis, psoriasis, eczema, seborrhea, insect bites, and contact dermatitis. The composition can be sprayed, rubbed, wiped, rolled or otherwise dispersed onto the skin or mucosa. Example steroids include, cortisone, fluocinolone, desonide, predicarbate, and fluticasone propionate.

In an embodiment with a burn or sunburn relief agent, the barrier-forming composition is used for relief of burn discomfort or to promote healing. The composition may be applied, for example, to the affected skin. The composition can be sprayed, rolled, rubber, wiped, or otherwise dispersed onto the skin. Example active agents include: aloe vera, steroids, and NSAIDS. An active agent such as beta-carotene and/or lycopene may be effective as a protective and preventive agent against sunburn.

In an embodiment with a counter-irritant agent, the barrier-forming composition is used for relief of skin or mucosal irritation. The composition may be applied, for example, to the nasal, oral, or pharyngeal mucosa or skin. The composition can be sprayed, rolled, wiped, rubbed, or otherwise dispersed onto the mucosa. Example anti-irritants include: nifedipine, alpha-bisabolol, canola oil, glycerol, hexapeptides, and those that treat pain or irritation by creating a feeling of warmth or coolness, e.g. camphor, eucalyptus, menthol, or wintergreen.

In an embodiment with a hair regrowth agent, the barrier-forming composition is used for hair regrowth, such as in treatment of androgenetic alopecia, alopecia areata, or more severe forms of alopecia (totalis or universalis). The composition may be applied, for example, to the scalp or other area of skin that hair generally grows, such as the face. The composition can be sprayed, rolled, wiped, rubbed, or otherwise dispersed onto the scalp. Example hair-regrowth agents include minoxidil, irritants such as cayenne pepper, ketoconazole, and finasteride.

In an embodiment with an erectile dysfunction agent, the barrier-forming composition is used for treating impotence in males or erectile dysfunction. The composition may be applied, for example, to the nasal, oral, or pharyngeal mucosa or skin in order to be absorbed into the bloodstream. The composition can be sprayed, rolled, wiped, rubbed, or otherwise dispersed onto the male genitals. The composition can be sprayed, rolled, wiped, rubbed, or otherwise dispersed onto the scalp. Example erectile dysfunction treatment agents include sildenafil, tadalfil, vardenafil, alprostadil and yohimbine.

In an embodiment with a wound treatment agent, the barrier-forming composition is used for relief of wound discomfort or to promote healing. The composition may be applied, for example, to the affected skin. The composition can be sprayed, rolled, rubber, wiped, or otherwise dispersed onto the skin. Example active agents include: NSAIDS, antimicrobials, and tissue regeneration agents. In an embodiment, a wound dressing may be treated with the barrier-forming composition just prior to application or may be packaged as a pre-treated wound dressing.

In an embodiment with an antacid active agent, the barrier-forming composition is used for treatment of acid reflux or an excess of acid. The composition may be applied, for example, to the throat or pharyngeal mucosa, or the oral mucosa, coating of the stomach may also occur if the barrier-composition is swallowed or expectorated. The composition can be sprayed or otherwise brought into the oral cavity and swallowed or expectorated. Example antacid active agents include calcium and magnesium carbonate, magnesium and aluminum hydroxide, sodium carbonate and bicarbonate, and C₇H₅BiO₄, and mixtures thereof.

In an embodiment with a pro-biotic active agent, the barrier-forming composition is used for treatment of various conditions such as intestinal inflammation, diarrhea, urogenital infections, infections or inflammation of the intestines, or allergies. The composition may be applied, for example, to the oral, nasal, anal, vaginal, or pharyngeal mucosa, coating of the stomach may also occur by swallowing the barrier-composition. The composition can be sprayed or otherwise brought into the oral cavity and swallowed or expectorated. Example pro-biotics include lactic acid bacteria (LAB) and bifidobacteria. Pro-biotic yeasts and bacilla are other examples. Pro-biotic active agents may not be compatible in an embodiment of the composition that also includes an antimicrobial agent, but they might be used in a separate embodiment after an antimicrobial is administered to restore beneficial microbes.

In an embodiment with a nutraceutical active agent, the barrier-forming composition is used for treatment or prevention of various conditions such as hypercholesterolemia, cancer, poor arterial health, cardiovascular disease. The composition may be applied, for example, to the oral, nasal, anal, vaginal, or pharyngeal mucosa, coating of the stomach may also occur by swallowing the barrier-composition. The composition can be sprayed or otherwise brought into the oral cavity and swallowed or expectorated. Example nutraceuticals include resveratrol from red grape products, flavonoids inside citrus, tea, wine, and dark chocolate, anthocyanins found in berries, soluble dietary fiber products, such as psyllium seed husk, broccoli (sulforaphane), fiddleheads (Matteuccia Struthiopteus), soy or clover (isoflavonoids), alpha-linolenic acid from flax or chia seeds, Omega 3 fatty acids in fish oil, botanical and herbal extracts such as ginseng, and garlic oil.

In an embodiment with an antioxidant active agent, the barrier-forming composition is used for treatment or prevention of various conditions such as heart disease and neurological conditions. The composition may be applied, for example, to the oral, nasal, or pharyngeal mucosa, coating of the stomach may also occur by swallowing the barrier-composition. The composition can be sprayed or otherwise brought into the oral cavity and swallowed or expectorated. Example anti-oxidants include thiols, carotene, ubiquinol, ascorbic acid, or polyphenols and may be either hydrophobic or hydrophilic.

In an embodiment cold and flu medicaments are active agents in the barrier-forming composition and the composition is used for treatment or prevention of cold and flu symptoms. The composition may be applied, for example, to the oral, nasal, or pharyngeal mucosa, coating of the stomach may also occur by swallowing the barrier-composition. Example medicaments include decongestants, anti-diarreahals, anti-nausea, and antihistamines, further details of which are listed herein separately.

In an embodiment with an anti-histamine agent, the barrier-forming composition is used for relief of mucous in the upper respiratory system. The composition may be applied, for example, to the nasal, oral, or pharyngeal mucosa, or in another embodiment in the eyes or ears. The composition can be sprayed, rolled, dropped, or otherwise dispersed onto the mucosa. Example anti-histamines include: azelastine, hydroxyzine, desloratadine, cyproheptadine, emadastine, levocabastine, carbinoxamine, levocetirizine, fexofenadine, diphenhydramine, brompheniramine, loratadine, clemastine, chlorpheniramine, and certirizine.

In an embodiment an anti-prion is an active agent in the barrier-forming composition and the composition is used for treatment or prevention of conditions of the brain, such as alzheimers, or other prion diseases, such as kuru or Mad Cow Disease. The composition may be applied, for example, to the oral, nasal, or pharyngeal mucosa, coating of the stomach may also occur by swallowing the barrier-composition. An example anti-prion is a quinoline or acridine ring, linked via tethers in a planar aromatic structure. This structure encompasses sulfated glycans or other compounds, such as trimesic acid.

In an embodiment with an immunostimulant active agent, the barrier-forming composition is used for stimulating the body's immune system. The composition may be applied, for example, to the oral, nasal, or pharyngeal mucosa, coating of the stomach may also occur by swallowing the barrier-composition. The composition can be sprayed or otherwise brought into the oral cavity and swallowed or expectorated. Example immunostimulants include specific, and non-specific immunostimulants, endogenous immunostimulants, deoxycholic acid, a stimulator of macrophages, synthetic immunostimulants, macrokine, imiquimod, resiquimod, and granulocyte macrophage colony-stimulating factor Immunostimulators may be particularly effective in combination with an antimicrobial in the composition to prevent disease.

In an embodiment with a vitamin active agent, the barrier-forming composition is used for treating vitamin deficiencies or otherwise providing the body with needed vitamins. The composition may be applied, for example, to the oral, nasal, or pharyngeal mucosa, coating of the stomach may also occur by swallowing the barrier-composition. The composition can be sprayed or otherwise brought into the oral cavity and swallowed or expectorated. Example vitamins include A, B, C, D, E, and K, and sub-scripted varieties of the same.

In an embodiment with an anti-diarrheal active agent, the barrier-forming composition is used for treating diarrhea. The composition may be applied, for example, to the oral, nasal, anal, vaginal, or pharyngeal mucosa, coating of the stomach may also occur by swallowing the barrier-composition. The composition can be sprayed or otherwise brought into the oral cavity and swallowed or expectorated. The composition can be applied to the anal mucosa in an enema applicator. Example anti-diarrheal active agents include, for example, anti-inflammatory solutions like bismuth subsalicylate, bulking agents like methylcellulose, guar gum or plant fibre (bran, sterculia, isabgol, absorbents such as methyl cellulose, opioids, and loperamide hydrochloride.

In an embodiment with an antiemetic active agent, the barrier-forming composition is used for treating nausea. The composition may be applied, for example, to the oral or pharyngeal mucosa, coating of the stomach may also occur by swallowing the barrier-composition. The composition can be sprayed or otherwise brought into the oral cavity and swallowed or expectorated. Example anti-nauseant active agents include, for example, olanzapine, 5-HT3 receptor antagonists, dopamine antagonists, dolasetron, NK1 receptor antagonist, aprepitant, H1 histamine receptor antagonists, cyclizine, diphenhydramine, cannabinoids, cannabis, dronabinol, benzodiazepines, midazolam, lorazepam, anticholinergics, hyoscine, steroids, and dexamethasone.

In an embodiment with a decongestant active agent, the barrier-forming composition is used for relief of excess mucous. The composition may be applied, for example, to the nasal, oral, or pharyngeal mucosa. The composition can be sprayed, rolled, or otherwise dispersed onto the mucosa. Example decongestants include: pseudoephedrine, phenylephrine, ephedrine, levo-methamphetamine, naphazoline, oxymetazoline, phenylpropanolamine, propylhexedrine, synephrine, and tetrahydrozoline.

In an embodiment with a cough suppressant agent, the barrier-forming composition is used for relief of coughing. The composition may be applied, for example, to the nasal, oral, or pharyngeal mucosa. The composition can be sprayed, rolled, or otherwise dispersed onto the mucosa. Example cough suppressants include: antitussives, dextromethorphan, codeine, noscapine, bromhexine, acetylcysteine, expectorants, mucolytics, and honey.

In an embodiment with an expectorant agent, the barrier-forming composition is used for relief of mucous in the upper respiratory system. The composition may be applied, for example, to the nasal, oral, or pharyngeal mucosa. The composition can be sprayed, rolled, or otherwise dispersed onto the mucosa. Example expectorants include: acetylcysteine and ambroxol, guinefesin.

In an embodiment, the barrier-forming composition with an active agent listed above also includes an antimicrobial component to inhibit microbial growth through static or cidal activity for an extended period of time. The combined barrier coating and antimicrobial synergistically act to block, neutralize, and/or kill microorganisms recently deposited on the treated surface and/or microorganisms that subsequently come into contact with an exposed (top) surface of the barrier coating, thereby providing a long-lasting antimicrobial that is significantly more powerful than just an antimicrobial alone. The barrier coating is effective to trap and kill or neutralize microorganisms already present on the treated surface and/or to trap and kill or neutralize microorganisms that are subsequently deposited on top of the barrier, i.e., the exposed surface of the barrier coating, after the administering of the barrier forming composition is performed.

Without being bound by theory, the mechanism of action of the barrier-forming composition with antimicrobial is based on a synergistic dual-action mechanism, in which germs are trapped in the formed barrier coating, and subsequently killed by the antimicrobial active ingredient. In an embodiment, the barrier-forming composition is not hydrophilic, which, without being bound by theory, is theorized to enhance its sustained effectiveness.

A similar dual action mechanism of sealing and acting on the skin or mucosal surface of a body may also be provided by the barrier coating with actives. The active agent has art-recognized activity on the surface and is sustained in contact with a surface for a long duration by including it in the barrier composition, thereby resulting in an increased duration on the body surface.

As shown in the Examples below, the properties of the composition with no actives, with antimicrobial only, and with antimicrobial and a homeopathic active were assessed using at least ten different approaches based on: (1) an in vitro anti-microbial susceptibility testing; (2) an in vitro time kill assay; (3) an in vitro biofilm model; (4) an in vitro filter insert-based model, (5) an in vivo-like engineered human oral mucosa (EHOM) model; (6) electron microscopy evaluation; (7) hydrophobicity assay; (8) physico-chemical compatibility assays; (9) cell culture-based model using monolayer of human cell lines; and (10) human clinical trials.

The barrier-forming composition is particularly useful for individuals that have an elevated risk condition causing the malady that the active ingredient is intended to treat. Due to the extended duration and improved efficacy of the active in the barrier coating, such persons may benefit from administration of the barrier-forming composition in repeated doses for ongoing proactive treatment. For example, the enhanced duration and efficacy of the composition may be especially useful for those with chronic pain or regularly exercise and have pain (pain medicament actives) or for those that have conditions that increase their need for the active agent on a regular basis, e.g. those with vitamin deficiencies (vitamin active agent).

The elevated risk condition may be due to an elevated risk of serious complications resulting from not treating the malady effectively, or an elevated risk of exposure to environmental conditions or contaminants that cause the elevated risk. Such as, for example, an elevated risk of allergic reaction due to allergen contaminants.

In an embodiment, the administration of the barrier-forming composition with active agent is in response to a recognized need for treatment with the active agent, as is typical for such active agents. In another embodiment, however, the administration may be in response to identification or expectation of a future need for the active agent's treatment, such as, for example, further in the future than is currently recognized in the treatment regimen time-frame for the active agent. The enhanced duration of the composition facilitates such use. For example, the enhanced duration facilitates use of the barrier-forming composition well before a recognized need occurs for an antacid, anti-allergen, anti-nausea, or topical muscle pain medication (NSAIDs, salicylates, steroids). Because of the duration of the barrier, the barrier-forming composition may be administered up to about 24 hours prior to the identified future need, such as for example, between about 1 hour to about 16 hours, about 2 hours to about 8 hours, or 6 hours to 10 hours. This enhanced time frame provides treatment options that were previously not possible.

In an embodiment, a method for treating a body surface with an active agent, includes administering a barrier-forming composition in a therapeutically effective amount to a body surface, the barrier-forming composition forming a barrier coating on the body surface that is active to sustain contact with the body surface for a duration of at least about one hour.

In an embodiment of the method, the step of administering the barrier-forming composition occurs in response to one of the following conditions: (a) identifying a future need for an active agent; and (b) observing an event that triggers a future need for the active agent. In an embodiment, the step of administering is performed in response to (a) or (b) and may be the first administration of the barrier forming composition to begin a multi-dose regimen that continues until the individual no longer has a need for the active agent. In an embodiment, second or subsequent therapeutically effective doses may be administered in response to conditions (a) or (b). For example, a person may first administer the barrier-forming composition when an observed event occurs, and then continues to administer the barrier-forming composition in the dosage intervals described above until the individual leaves the vicinity of the contamination event. In another example, after a dosage regimen has begun, the individual may administer a second or subsequent dose when a new contamination event is observed, so long as the administration occurs no sooner than the minimum of the described dosing time interval.

In an embodiment, an observed event includes a contamination event. A contamination event, includes, for example, an individual sneezing, coughing, or vomiting, or more generally where bodily fluids or matter have been deposited. It also includes a dispersion of allergens into the air or onto a surface. In an embodiment, the contamination event is in the vicinity of the individual to trigger the administering response. The vicinity of the individual may be defined as being in the same room, vehicle, or within about 10 yards of the individual.

In an embodiment, the barrier-forming composition is applied to a mucosa of an individual with an elevated risk condition. The mucosa, may be an oral, pharyngeal, or nasal mucosa. In an embodiment, the administering step is performed in response to encountering an environment that is considered to be contaminated or in response to an observed contamination event. The barrier-forming composition provides a barrier coating on the mucosa surface that provides sustained activity of the active agent. In an anti-allergen embodiment, the barrier-forming composition may prevent or inhibit allergens from passing to the mucosa or causing a reaction.

In another embodiment, a barrier-forming composition is administered in a method of treating a mammal with a disrupted mucosa or skin lesion, such as for example an immunocompromised mammal. The disrupted area or lesion of the mammal is identified and a therapeutically effective amount of a barrier-forming composition is administered to at least the disrupted area of the mucosa of the mammal. The barrier-forming composition with an antimicrobial provides a barrier on the disrupted area of the mucosa that effectively inhibits active microorganisms from disseminating to a disrupted area of the mucosa. Other active agents that are safe for application to open wounds may be included to promote healing.

In an embodiment, the topical antimicrobial composition or the barrier-forming composition with antimicrobial and other actives is used in a method of preventing an infectious disease. A step includes identifying a contaminated environment or item that a mammal is expected to encounter. The contaminated environment is an environment such as an indoor or outdoor space or a proximity to another mammal or human that is known or expected to be contaminated with harmful viral, fungal, or bacterial microorganisms. The determination of whether a given environment may be contaminated may be based on the time of year, published information on flourishing diseases in the community, or observing others that appear to be sick or spreading germs by sneezing, etc. The latter factor may also be described as observing a contamination event.

Predicting or identifying whether the contaminated environment or item will be encountered can be a decision based on whether the mammal plans expects, or is expected to enter the environment or encounter the item in the near future. This may include estimating a time when the contaminated environment or item will be encountered. The composition may then be administered about twenty-four hours or less prior to the estimated time of encounter with the contaminated environment or item, such as, for example, about sixteen hours or less, about twelve hours or less, about six hours or less, or about two hour or less. The composition sets up quickly and should be operable to prevent or inhibit harmful microorganisms from infecting mucosa, for example, within less than one minute of application, such as within 30 seconds. Thus, it could be applied during the encounter with the contaminated environment or item and have effectiveness.

In an embodiment, the topical antimicrobial composition is utilized to kill germs on the skin of a mammal. The composition may be applied by spraying, rubbing, or otherwise distributing the composition on a topical surface such as, for example, the hands, arms, feet, legs, face, or genitalia. In an embodiment, the composition is applied after a known or suspected contact with a contaminated environment or item. In an embodiment, the composition is applied daily or several times a day as a hand sanitizer, such as before meals, and after using the restroom. In an embodiment, the composition is applied to the skin prior to entering an environment that must be clear of harmful microorganisms, such as an operating room, or prior to participating in activities such as surgery, invasive body treatments, child-birth, dental procedures, pre-operation cleaning, or wound treatment. In an embodiment, the skin surface is a pre-designated surgical site. In an embodiment, the skin surface does not include the genitalia.

In an embodiment, the composition and method of treatment and prevention described herein may be useful, for example, for prevention of infections in environments such as hospitals and infections common in such environments that are contaminated with infectious microorganisms. As mentioned above, the methods and compositions disclosed herein may be especially applicable for immunocompromised patients or persons that spend significant working hours in health-care facilities. In addition, the composition may be useful for prevention of infections by microorganisms that commonly infect wounds.

The contaminated environment may include, for example, a public transportation vehicle, a public gathering place, and a room or vehicle containing a mammal known or expected to be ill, or a close proximity to a mammal known or expected to be ill. More information on environments commonly recognized as contaminated environments, such as an airplane, a nursery, and a health center, is disclosed in Yang, et al., “Concentrations and Size Distributions of Airborne Influenza A Viruses Measured Indoors at a Health Centre, a Day-Care Centre, and on Aeroplanes,” J.R. Soc. Interface (Feb. 7, 2011), which is incorporated herein by reference.

More specifically, in an embodiment, the public transportation vehicle may be, for example, an airplane, a bus, or a taxi. A public gathering place may be, for example, a doctor's office, a hospital, a school, a nursery, a church, a hotel, or a restaurant. The close proximity to a mammal known or expected to be ill may be, for example, within a one foot radius, or in the same motor vehicle with the mammal. A publicly used airplane may be mentioned as a common and particularly noteworthy example of an environment that many would identify as being a contaminated environment. As such persons that work on airlines or travel on public airplanes very frequently, e.g. two or three times per week, may be considered to have the elevated risk condition due to increased exposure to microorganisms.

In an embodiment, in a continued dosage method of prevention or treatment, the barrier-forming composition may be administered in a therapeutically effective amount in a series of doses so long as it is consistent with the prescribed dosage regimen of the particular active agent used in the barrier-forming composition. The continued dosage regimen, for example, may be once every 1 to 12 hours, about every 2 to 8 hours, or about every 4 to 6 hours. In another embodiment, the therapeutically effective amount of the barrier-forming composition is administered every about two to about twelve hours to the surface, such as every about three to about eight hours, or every about four to about six hours. Administering “every about two to about twelve hours” means one therapeutically effective dose being administered and then a second dose being administered about two hours later up to about twelve hours later, and additional doses, if taken, being administered in subsequent about two hour to about twelve hour increments. The topical antimicrobial composition may be applied in the same dosages. The dosage regimen may be continued until the therapeutic effect is no longer needed.

In an embodiment, the barrier forming composition is administered in a therapeutically effective amount three times or more in a 24 hour period for five 24 hour periods or more, such as, for example, four to twelve times, or six to ten times for six days to ten days, or seven to thirty days. In another embodiment, the method of prevention can be continued, for example, for a day or more, such as for about two days to about a week. In an embodiment, the three or more doses may be taken only during daylight hours or an individuals waking hours, such as, for example, 6 AM to 6 PM, or 9 PM to 5 PM. In an embodiment, an individual may follow such a dosage regimen to provide protection during the entirety of their hours at a workplace or another public gathering place. In an embodiment, the continued dosage method may be preferred when the subject is in prolonged contact with a contaminated environment or item.

The body surface treated will vary according to the active agent that is in the barrier-forming composition. Mucosa that is treated, may, for example, may be a mucosal surface in the oral cavity, the nasal cavity, throat, or the pharyngeal cavity, such as, the nasopharynx (epipharynx), the oropharynx (mesopharynx), or the laryngopharynx (hypopharynx). Other mucosa in other orifices of a mammal, may also be treated, including, for example, the vaginal, anal, or ear canal. Skin or other body surfaces, such as, for example, the sites of sore muscles, infections, wounds, acne, scalp, eyes, may also be treated, based on the active agent and its typical application area. In an embodiment, the composition is administered only to skin, and not to mucosa or taken internally.

In an embodiment, the composition is administered to a skin or mucosal lesion in response to identification of the skin or mucosal lesion. Subsequent dosages may be applied in accordance with dosage intervals discussed above. In an embodiment, the dosing regimen is ended when the lesion has healed, i.e. when it is covered with new skin or mucosal tissue.

In an embodiment, the barrier-forming composition traps and/or kills or neutralizes all harmful microorganisms contacting the barrier-forming composition. In another embodiment, the barrier substantially traps and/or kills or neutralizes enough harmful microorganisms that contact the barrier-forming composition to inhibit or even stop them from causing an infectious disease. In the latter case, if the harmful microorganism's penetration of the barrier is slowed and/or diluted it will enhance the body's own ability to prevent the microorganisms from causing disease or widespread infection. The addition of other active agents, such as steroids may enhance the effectiveness of the barrier-forming composition with antimicrobial. In the topical antimicrobial composition a barrier need not necessarily even form for an effective skin antimicrobial.

In vitro testing demonstrates that embodiments of the barrier-forming composition prevent all active bacteria from reaching the other side of the barrier for long periods, including about two hours or more, about six hours or more, about sixteen hours or more, and about twenty-four hours or more. In vitro testing shows that in viruses exposed to embodiments of the barrier-forming composition, growth may be inhibited for about two or more days (such as influenza), up to about nine days, (such as HIV), after which the viral count is still below the MIC for extended periods, such as about two or three additional days. Inhibitory activity against influenza virus was observed for up to 48 hours.

The barrier-forming composition exhibited the activity to reduce the microbial load of humans in clinical trials. For example, a surprisingly effective reduction in microbial load of more than about 50% to about 99% from about one to about six hours after the administering step was demonstrated. In embodiments, the microbial load may be reduced by more than about 10%, by more than about 25%, or by more than about 70% from about one to about six hours after the administering step. Furthermore, these ranges of reduction in microbial load are sustainable for long periods of time with the disclosed dosing regimen.

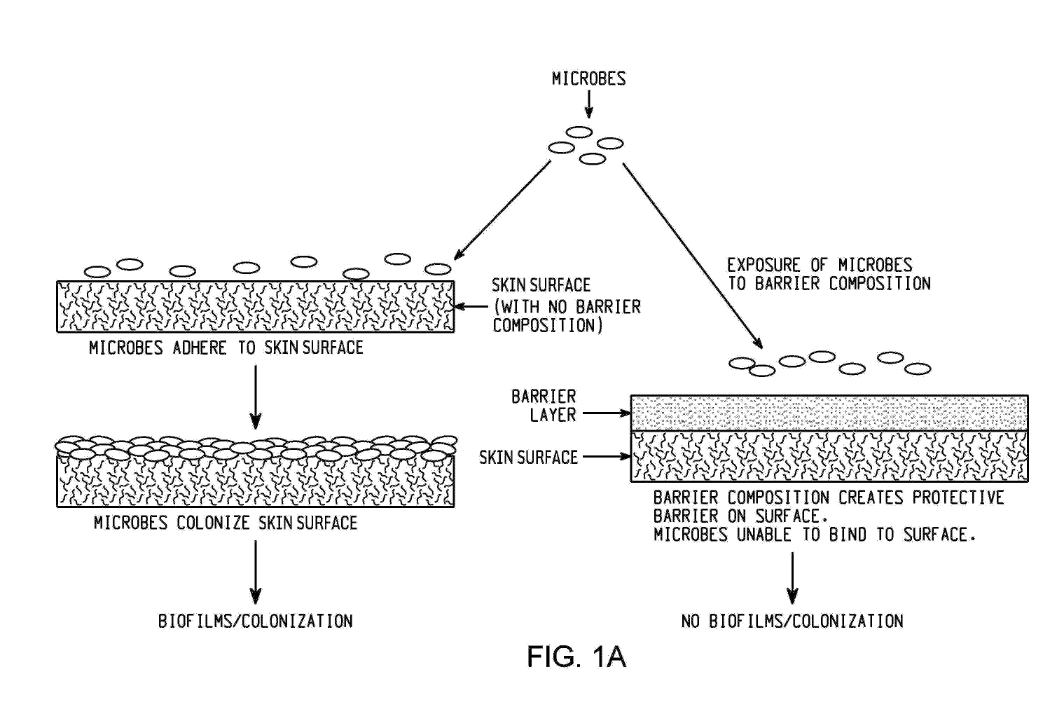

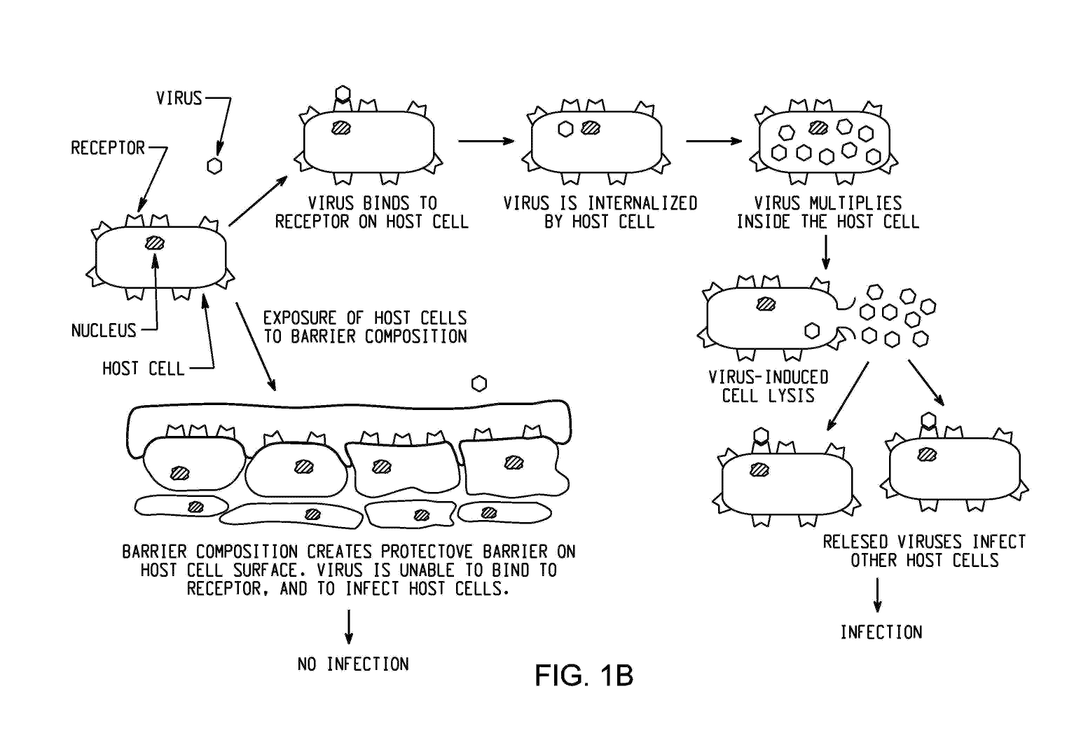

FIG. 1A is an illustrated flow chart of microbes encountering an untreated skin surface (left side) and a skin surface with the coating composition administered on it resulting in a formed coating layer (right side) that shows a primary efficacy of the coating composition on a skin surface. Instead of protecting a mucosa from infection from microbes, when treating an skin surface, the coating layer kills microorganisms on the surface and prevents microorganisms from binding to the surface, colonizing, and forming a biofilm. Biofilms are known to be difficult to destroy. The coating composition thus presents a surprisingly effective solution to providing a sanitized skin surface in comparison to cleaners that only focus on a quick and short-duration kill of microorganisms that are only already on the surface. While an antimicrobial solution that does not form a barrier coating will instantly kill some of the microorganisms in a biofilm on a surface, it is practically impossible to kill all microorganisms in a biofilm and the biofilm will soon begin to recolonize. In an embodiment, the coating composition prevents biofilms from forming in the first place and also has prolonged activity to destroy already formed biofilms. While the low tack embodiment of the coating composition may not be as robust at preventing passage of microorganisms as other embodiments with more humectant and carbohydrate gum, it was found to be effective in killing or neutralizing the microorganisms and was shown to have potent activity against MRSA and Candida.

In an embodiment that illustrates a proposed mechanism of the barrier-forming composition, shown in FIG. 1, the barrier-forming composition provides anti-viral activity. When a virus comes into contact with a cell, it will bind to receptors on the host cell. Over time, 5 to 6 hours, or so, the virus is internalized by the host cell, the virus multiplies inside the host cell, and it induces cell lysis causing additional virus particles to infect other host cells.

In contrast, in a cell treated with the barrier-forming composition, a protective barrier is on the surface of the host cell. The barrier, which is thick enough to cover the cell and any receptors on the cell, prevents the virus particle from binding to the cell receptors. Thus, infection and lysis is also prevented. The barrier-forming composition retains the barrier for a long duration, such as a duration of about 1 hour of more, a duration of about 2 hours or more, a duration of about 6 hours or more, a duration of about 16 hours or more, a duration of about 16 hours to about 24 hours, or a duration of about 24 hours or more, thereby protecting host cells and preventing infection. The cidal or static antimicrobial activity is also retained for a long duration, such as about 2 hours or more, about 6 hours or more, about 16 hours or more, about 24 hours or more, or about 48 hours or more, thereby killing microorganisms and reducing microbial load. These durations are applicable for viruses, bacteria, and fungi.

Harmful microorganisms are those known to cause infectious disease such as, for example, such as communicable diseases caused by microorganisms, such as Candida species (e.g. C. albicans, C. glabrata, C. krusei, C. tropicalis), Staphylococcus species (including methicillin-resistant S. aureus, MRSA), Streptococcus species (e.g. S. sanguis, S. oralis, S. mitis, S. salivarius, S. gordonii, S. pneumoniae), Acinetobacter baumannii, Aggregatibacter actinomycetemcomitans, Fusobacterium nucleatum, and other microorganisms such as microorganisms that cause upper respiratory infections, and common cold (rhinovirus) and influenza viruses and Pneumonia, P. gingivalis, Y. enterocolitica, Acinetobacter bumanii, Aggregatibacter actinomycetemcomitans, microorganisms that cause odor, microorganisms that can detract from visual appeal of surfaces, Clostridium difficile, Bordetella pertussis, Burkholderia, Aspergillus fumigatus, Penicillium spp, Cladosporium, Klebsiella pneumoniae, Salmonella choleraesuis, Escherichia coli (0157:H7), Trichophyton mentagrophytes, Rhinovirus Type 39, Respiratory Syncytial Virus, Poliovirus Type 1, Rotavirus Wa, Influenza A Virus, Herpes Simplex Virus Types 1 & 2, and Hepatitis A Virus. In an embodiment, the barrier-forming composition and method of treatment and prevention described herein may be useful, for example, for prevention of sexually transmitted diseases such as, for example, infections caused by human immunodeficiency virus (HIV), Herpes simplex, or human papilloma virus (HPV).

Most of the above species of microorganisms have been tested and found to be effectively inhibited or killed by the barrier-forming composition. Furthermore, the barrier-forming composition has shown effectiveness against microorganisms with a diameter of, for example, about 30 nm or greater, such as about 100 nm (HIV, spherical), about 100 to about 300 nm (influenza, spherical and elongated forms), about 120 nm to about 260 nm (EBV spherical/disk forms), and about 30 nm (rhinovirus, spherical). Thus, the barrier composition should also be effective against other microorganisms with diameters of about 30 nm, or greater than about 30 nm.

The composition has even shown powerful and surprising activity inhibiting biofilms, which can be very difficult to eradicate. In an embodiment, the method comprises administering the barrier-forming composition to a formed biofilm on a mucosa or lesion.

The microorganisms may be air-borne microorganisms. In an embodiment, the microorganisms are those that cause communicable diseases. In an embodiment, the microorganisms do not include those that cause allergic reactions or dental problems, such as, for example, cavities (caries), gingivitis, or seasonal allergies. Similarly, in an embodiment, the method of prevention does not solely or additionally prevent dental problems or allergic reactions, such as, for example, cavities (caries), gingivitis, or seasonal allergies.

In another embodiment, however, microorganisms, such as fungi that may generally be classified as allergens, other allergens, and airborne irritants to the mucosa, are also blocked by the barrier-forming composition and the method.

A therapeutically effective amount of the total barrier composition with active agent includes an amount that is enough to coat the targeted mucosa or lesion with the barrier-forming composition to form a barrier coating that will result in a barrier layer forming on the mucosa or lesion. For example, about 100 microliters to about 10 ml, such as, for example, about 1 ml to about 8 ml, or about 2 ml to about 5 ml for a mouthwash formulation, or about 0.125 ml to about 2 ml, such as about 0.5 ml to about 1 ml for a spray formulation. The dosage amount may also be expressed in terms of a volume per square cm, such as, for example, from about 0.5 to about 50 μl/cm², such as, about 5 to about 40 μl/cm², or about 10 to about 25 μl/cm² for a mouthwash formulation; or for a spray formulation, for example, about 0.625 to about 10 μl/cm², such as, about 2.5 to about 5 μl/cm². Other delivery mediums, such as dissolvable strips, may have dosages derived from these ranges given the adjustments for concentrations and other factors known to those of skill in the art. In addition, the average thickness of the film formed on the mucosa from the barrier-forming composition may range, for example, from about 0.001 to about 0.2 mm, such as about 0.01 mm to about 0.1, or about 0.08 to about 0.15 mm. For example, for a given human or animal, the therapeutically effective amount can be determined based on the age or weight or size of the mammal to be treated, and the dosage may be those listed above. For non-human mammals, in particular, the dosage amount may be adjusted according to the per square cm values given above and the approximate surface area of the mucosal surface or body cavity to be treated. The topical antimicrobial composition may be applied in the same quantities on skin surfaces.

Other delivery mediums, such as a liquid filled lozenge or roll-on applicator, or treated wiping material may have dosages derived from these ranges given the adjustments for concentrations and other factors known to those of skill in the art.

The average thickness of the film or coating formed on the mucosal, skin, or skin lesion surface from the barrier-forming composition may range, for example, from about 0.001 to about 0.2 mm, such as about 0.01 mm to about 0.1, or about 0.08 to about 0.15 mm.

A mechanical pump spray or an aerosolized spray device may be used. In the aerosolized embodiment, the barrier-forming composition may be mixed with common propellant agents, such as CO₂, nitrogen, and hydrocarbons. A bag-on-valve embodiment may also be used; however, the composition is stable enough so as not to require a separation of the propellant agent and the composition components.

As the Examples below show, the barrier-forming composition has been shown to block and trap, and kill or neutralize a wide variety of representative fungi, bacteria and viruses.

Several experiments were performed to assess the safety of the composition on mammals and the ability of the spray formulation to form a protective barrier on an Engineered Human Oral Mucosa (EHOM) model and on pig skin. The experimental evidence showed that the composition formed a barrier coating over tissues and skin, which prevents microorganisms from penetrating into the tissues and provides a long-lasting coating.

### EXAMPLES

### Example 1

### Human Gingival Epithelial Cell and Fibroblast Cultures

Normal human gingival cells (epithelial cells and fibroblasts) were obtained from ScienCell Research Laboratories (Carlsbad, Calif., USA). The fibroblasts were cultured in Dulbecco's modified Eagle's medium (DME, Invitrogen Life Technologies, Burlington, ON, Canada) supplemented with fetal bovine serum (FBS, Gibco, Burlington, ON, Canada) to a final concentration of 10%. The epithelial cells were cultured in Dulbecco's modified Eagle's (DME)-Ham's F12 (3:1) (DMEH) with 5 μg/mL of human transferrin, 2 nM 3,3′,5′ of tri-iodo-L-thyronine.

0.4 μg/mL of hydrocortisone, 10 ng/mL of epidermal growth factor, penicillin and streptomycin, and 10% FBS (final concentration). The medium was changed once a day for epithelial cells and three times a week for fibroblasts. When the cultures reached 90% confluency, the cells were detached from the flasks using a 0.05% trypsin-0.1% ethylenediaminetetra acetic acid (EDTA) solution, washed twice, and resuspended in DMEM (for the fibroblasts) or DMEH-supplemented medium (for the epithelial cells).

### Example 2

### Engineered Human Oral Mucosa (EHOM) Tissue

The EHOM model was produced by using the gingival fibroblasts and epithelial cells of Example 1 that were used to form a complex three-dimensional spatial cellular organization similar to that found in normal human oral mucosa. The lamina propria was produced by mixing Type I collagen (Gibco-Invitrogen, Burlington, ON, Canada) with gingival fibroblasts, followed by culture in 10% FBS-supplemented medium for four days. The lamina propria was then seeded with gingival epithelial cells to obtain the EHOM. The tissue specimens were grown under submerged conditions until the total surface of the lamina propria was covered with epithelial cells. To produce stratified epithelium, the EHOM was raised to an air-liquid interface for four more days to facilitate the organization of the epithelium into its different strata.

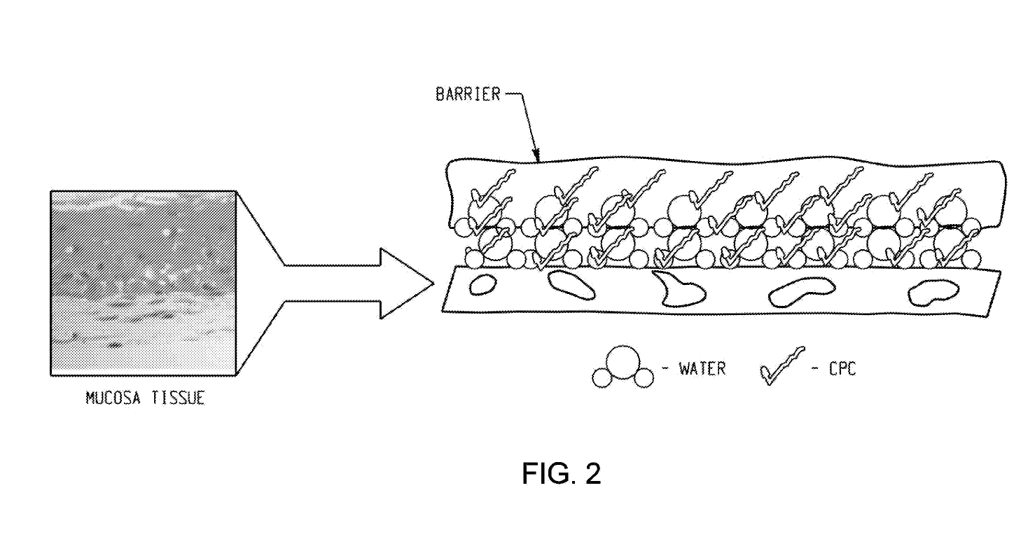

The lamina propria is a thin layer of loose connective tissue that lies beneath the epithelium and together with the epithelium constitutes the mucosa. FIG. 2 shows an illustration of the EHOM mucosal tissue, with an arrow pointing to its location in a schema depicting mucosa covered with the coating composition.

### Examples 3-9

Examples of the coating compositions were created by adding the ingredients listed below in a 50-mL centrifuge tube, and vortexing to bring to “free-flow” consistency. The constituents of the compositions and their approximate amounts are given in Table I (the values in Table I are percentages by weight of the total composition):

TABLE I

| | | | Example 5 | Example 6 | | | |
|---|---|---|---|---|---|---|---|
| | Example 3 | Example 4 | (control) | (control) | Example 7 | Example 8 | Example 9 |
| Glycerin | 7 | 35 | 35 | 35 | 35 | 7 | 7 |
| Xanthan Gum | 0.01 | 0.4 | 0.4 | 0.4 | 0.4 | 0.01 | 0.01 |
| Cetyl | 0.05 | 0.05 | | | 0.1 | 0.06 | 0.05 |
| Pyridinium | | | | | | | |
| Chloride | | | | | | | |
| Preservatives | No | No | No | Yes | Yes | Yes | Yes |

*Purified water comprised the remaining portion of the composition.
**Preservatives included methylparaben (0.1%), propylparaben (0.1%), sodium benzoate

(0.5%)

Based on the results below, the preservatives were found to be superfluous to the barrier coating formation and antimicrobial activity.

### Examples 10-26

Examples 10-26 were performed to demonstrate safety of the composition on mucosal surfaces. Prior patent publication U.S. 2012/0270909, as well as the provisional applications that this application claims the benefit of priority of, include this information.

### Examples 27 and 28

### Determination Whether the Coating Composition Affects Mechanical Barrier Function of EHOM Against Microbial Passage Through Mucosal Tissue

In Examples 27 and 28, two approaches were used to determine whether the control Examples formed a barrier coating that blocked the microbial passage through the mucosal tissues and also had an inherent anti-microbial effect. Growth in pass-through chamber and growth on EHOM surface was assessed by evaluating growth in agar media.

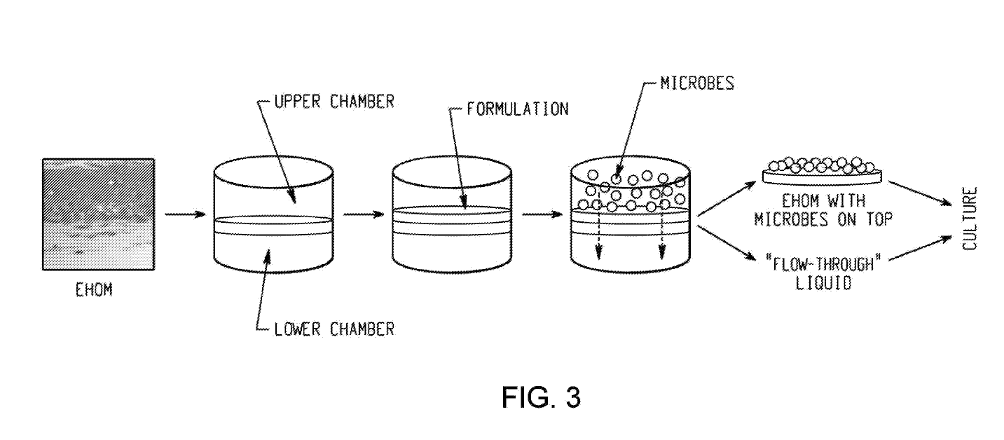

In Example 27, EHOMs of Example 2 were put in contact with 1 and 5% dilutions (diluted in serum free culture medium) of Example 4 for 2 minutes. Tissues were then washed twice with serum free culture medium then over layered with 1×10⁶ candida microbial cells in a volume of 300 μl. Tissues were then put on air-liquid culture plates and incubated for 24 hours in 5% CO₂ humid atmosphere at 37° C. Next, the culture medium underneath the EHOM (ventral chamber) was collected and seeded on Sabouraud agar plate to verify whether or not the microorganisms penetrated through the tissue and reached the culture medium below. A culture was also obtained from the EHOM surface and seeded on Sabouraud agar plate. The process is graphically depicted in FIG. 3.

In Example 28, EHOMs of Example 2 that were treated with 1 and 5% dilutions of the Example 4 composition for 2 minutes were over layered with candida microbial cells for 24 hours were flipped onto Sabouraud dextrose agar plates and left in place for 5 minutes. The EHOMs were then removed and the plates were incubated for 24 hours at 30° C., after which microbial growth was ascertained macroscopically and photographed. Each experiment was repeated 5 independent times with similar results.

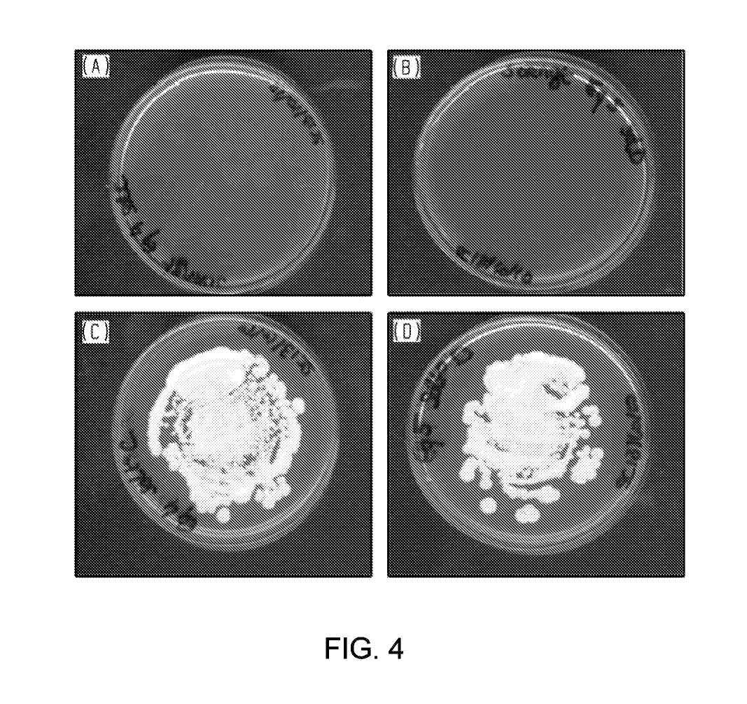

FIG. 4 shows the results of the cultures of the EHOM surface (panels C and D) and the culture of the pass-through liquid from the bottom (ventral) chamber (panels A and B). The A and C panels were EHOMs treated with a 1% dilution of Example 4, and the B and D panels were EHOMS treated with a 5% dilution of Example 4. This data indicates that Example 4 composition forms a barrier coating that prevents passage of microbes through the EHOM tissues but does not have an inherent anti-microbial effect.

### Examples 29 and 30

In Examples 29 and 30, Examples 27 and 28 were repeated, except the EHOM were infected with S. mutans. Similar results were obtained that indicated that the coating compositions formed a barrier coating preventing the S. mutans microbes from passing through the barrier coating, but did not have an antimicrobial effect.

### Examples 31 and 32

### Determination Whether the Coating Composition Affects Mechanical Barrier Function of EHOM Against Microbial Invasion

In Example 32, a set of EHOM tissues from Example 2 was treated with the coating composition of Example 4 and then overlaid with C. albicans. In control Example 31 a control set was not treated with the coating composition prior to overlayering with C. albicans Immediately after each contact period, biopsies were taken from each EHOM, fixed with paraformaldehyde solution, and embedded in paraffin. Thin sections (4 μm) were stained with eosin-hematoxylin. Sections were observed using an optical microscope to analyze the invasion/penetration of microbial cells into the tissue. Following microscopic observations, representative photos were taken from each condition and presented. The experiment was repeated three times with similar results. Similar results were also obtained with treatment with Example 3 (data not shown).

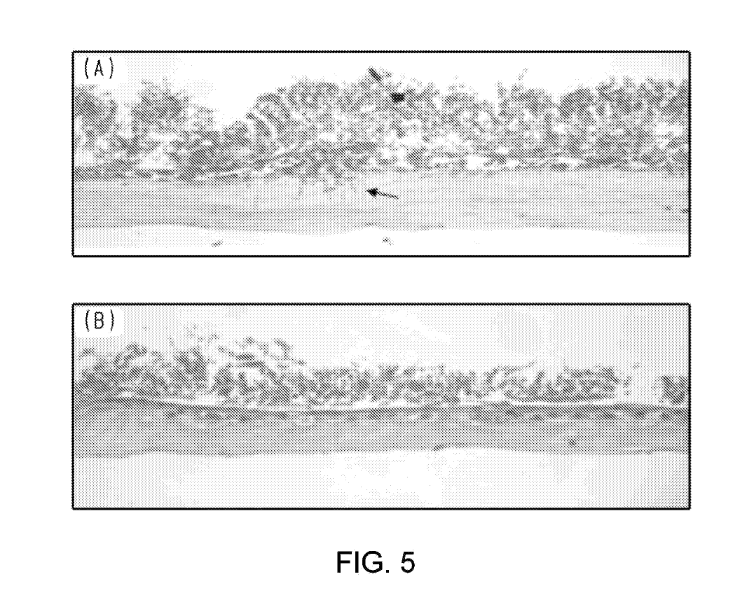

FIG. 5 shows the effect of the coating composition on microbial invasion of EHOM tissues. Panel (A) is a representative photograph of the untreated control Example 31, and panel (B) is a photograph of the treated Example 32. The arrow indicates invading fungal hyphae in the untreated control Example 31.

### Examples 33-40

The EHOM model described above was also used to evaluate the ability of Examples 5-7 to form a barrier coating that: (a) prevents oral bacteria (S. mutans) and fungi (Candida albicans) from penetrating/invading human oral mucosa, and (b) does not cause damage to host cells (cytotoxicity assay).

Examples 33-40 were formulated according to Table II below.

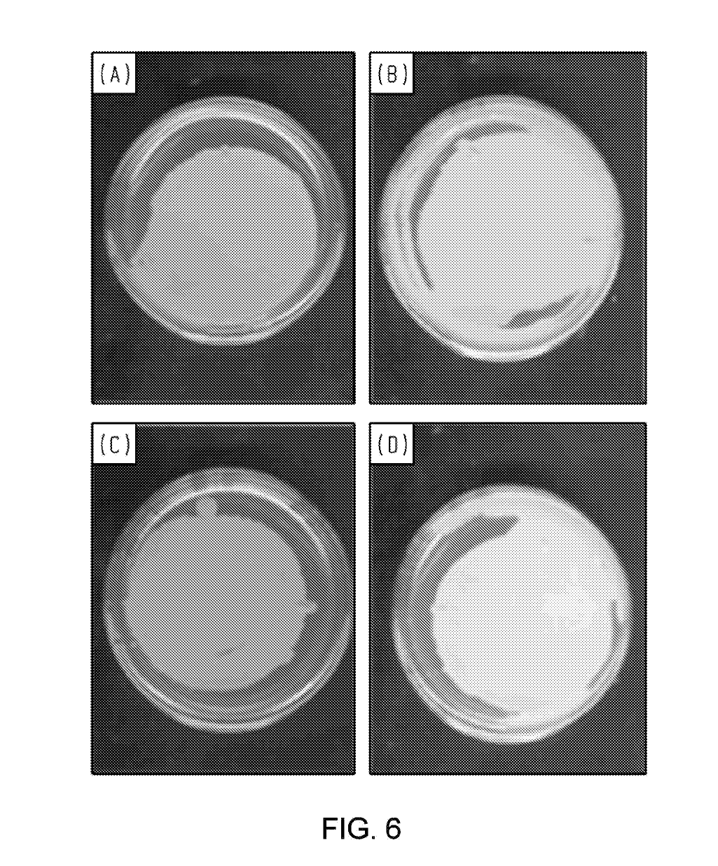

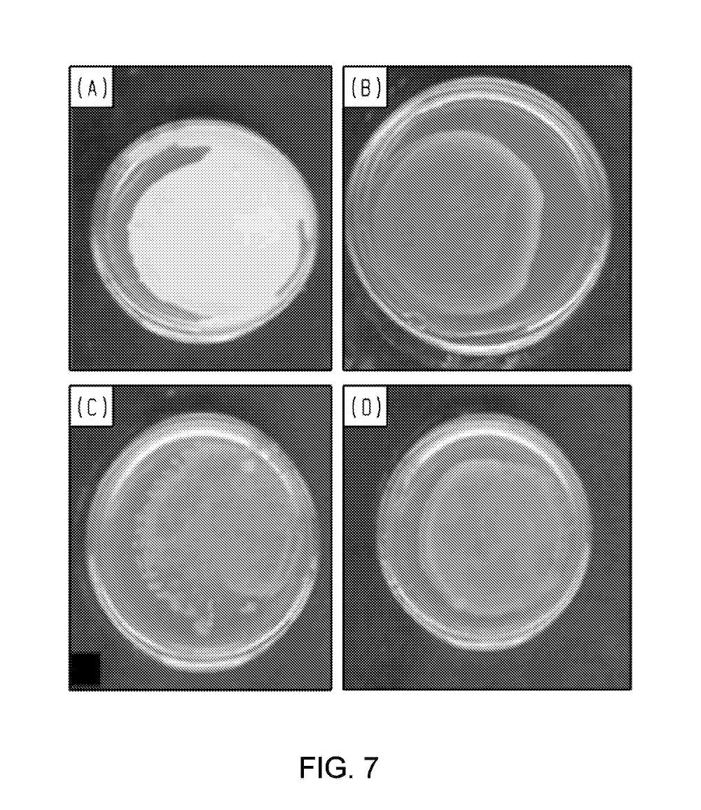

TABLE II

| | Coating composition | | Figure |
|---|---|---|---|
| | Pre-Treatment | Microbe Overlay | reference |
| Example 33 | None | C. albicans | FIG. 6(A) |
| Example 34 | Example 5 | C. albicans | FIG. 6(B) |
| Example 35 | Example 6 | C. albicans | FIG. 6(C) |
| Example 36 | Example 7 | C. albicans | FIG. 6(D) |
| Example 37 | None | S. mutans | FIG. 7(A) |
| Example 38 | Example 5 | S. mutans | FIG. 7(B) |
| Example 39 | Example 6 | S. mutans | FIG. 7(C) |
| Example 40 | Example 7 | S. mutans | FIG. 7(D) |

In Examples 33-40, after pre-treatment and incubation according to the procedures of Examples 27 and 28: (1) the flow-through medium was collected from the lower chamber; and (2) tissues were flipped and placed onto the surface of Sabouraud dextrose agar Petri dishes, and incubated for 24 hours. Collected flow-through media were spread onto agar media plates, and incubated for 24 hours also as described in Examples 27 and 28. Table II also indicates the figure in which a photo of each Example was taken showing the microbial growth on each flipped Example culture.

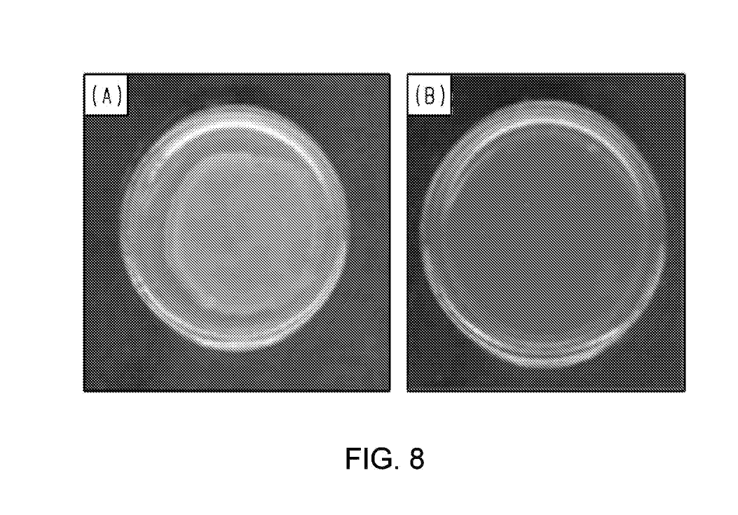

FIGS. 6 and 7 show that both Candida and Streptococcus were able to grow on the surface of EHOM treated with the compositions of Examples 5-6. In contrast, as shown in FIG. 8, no microbial growth was observed when the “flow-through” medium collected from the lower chambers of EHOMs of Examples 36 or 40, i.e. those treated with the Example 7 composition. This indicates that treatment of the EHOMs with the Example 7 composition did not cause damage to the surface of the mucosal tissues and organisms were unable to penetrate the treated EHOM. Similar results were obtained with EHOM treated with the compositions of Examples 5 and 6 (data not shown). These data indicate that the combination of glycerine and xanthan gum is capable of forming a protective barrier coating on mucosal tissues.

### Examples 41-47

### Tested Formulations are not Toxic and do not Cause Damage to the Cells/Tissues

In Examples 41-47, the EHOM model was used to assess the toxicity of the composition. Examples 41-47 were formulated as stated in Table III.

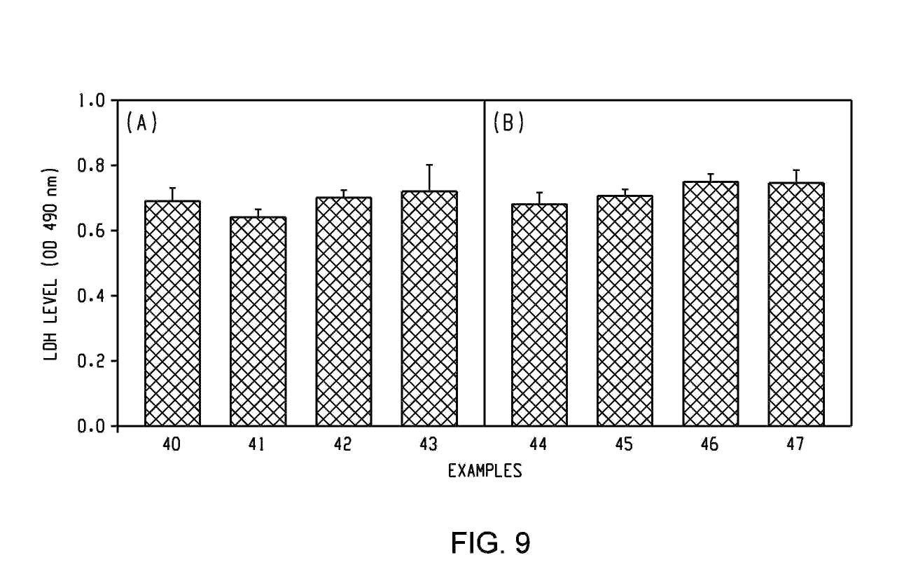

TABLE III

| | Coating composition | Microbe | |
|---|---|---|---|
| | Pre-Treatment | Overlay | Figure Reference |
| Example 41 | None | C. albicans | FIG. 9(A) |
| Example 42 | Example 5 | C. albicans | FIG. 9(A) |
| Example 43 | Example 6 | C. albicans | FIG. 9(A) |
| Example 44 | Example 7 | C. albicans | FIG. 9(A) |
| Example 41A | None | S. mutans | FIG. 9(B) |
| Example 45 | Example 5 | S. mutans | FIG. 9(B) |
| Example 46 | Example 6 | S. mutans | FIG. 9(B) |
| Example 47 | Example 7 | S. mutans | FIG. 9(B) |

After pre-treatment and incubation according to the procedures of Examples 27 and 28, culture supernatant was collected from the Example 41-48 EHOM tissues and used to measure LDH activity.

As shown in FIG. 9, no significant increase in LDH levels was observed in Examples 41-48 irrespective of whether the formulations contained cetylpyridinium chloride with or without preservatives and infected with either Candida albicans or S. mutans, respectively. These data confirmed the non-toxic effect of the example coating compositions and that these formulations maintained the integrity of the host mucosal tissues.

Data are mean±SD. No significant difference between untreated and treated tissues was noted.

Taken together, the data indicates that the example compositions represent an effective and a safe barrier that can prevent microorganisms from penetrating and invading human mucosal tissues.

### Examples 48-61

Preclinical evaluation of the coating composition showed that the composition was effective against many bacteria and yeasts. The antimicrobial activities of the Example 7 coating composition were evaluated against a number of clinical isolates obtained from patients, including S. salivarius, P. gingivalis, S. pyogenes, S. pneumonia, Fusobacterium nucleatum, S. mutans, S. aureus, Y. enterocolitica, S. oralis, S. mitis, C. albicans, C. krusei, C. tropicalis, and C. glabrata. Activity of the Example 7 coating composition was evaluated by determining its minimum inhibitory concentration (MIC) using reference methods described in the Clinical and Laboratory Standards Institute (CLSI) documents M07-A8, M11-A7, and M27-A3.

A standardized inoculum of several types of aerobic or anaerobic bacteria (1×10⁴ cells/ml) was incubated with serially diluted solutions of Example 7 (containing 0.1% CPC, or 1 μg/ml) or 2% chlorhexidine gluconate (CHX, 20 μg/mL) as a comparative example. Cells were allowed to grow in the presence or absence (growth control) of the test agents for 24 hours. The MIC for each agent was defined as the concentration that induced a 100% growth inhibition (compared to no-drug control).

A similar microdilution-based CLSI method (M27-A2) was used to evaluate the activity of Example 7 against albicans and non-albicans Candida species.

TABLE IV

| | | Example 7 | |
|---|---|---|---|
| | | MIC | Chlorhexidine MIC |
| | Organism | (μg/mL CPC) | (μg/mL chlorhexidine) |
| Example 48 | S. salivarius | 0.98 | 19.6 |
| Example 49 | P. gingivalis | 0.98 | 19.6 |
| Example 50 | S. pyogenes | 0.98 | 19.6 |
| Example 51 | S. pneumonia | 0.98 | 19.6 |
| Example 52 | F. nucleatum | 1.95 | 19.6 |
| Example 53 | S. mutans | 1.95 | 19.6 |
| Example 54 | S. aureus | 3.91 | 19.6 |
| Example 55 | Y. enterocolitica | 3.91 | 19.6 |
| Example 56 | S. oralis | 500 | 19.6 |
| Example 57 | S. mitis | 500 | 19.6 |
| Example 58 | C. albicans | 0.25 | 19.6 |
| Example 59 | C. krusei | 0.06 | 19.6 |
| Example 60 | C. tropicalis | 0.06 | 19.6 |
| Example 61 | C. glabrata | 0.125 | 19.6 |

The coating composition was also found to have potent antimicrobial activity against: MRSA, Acinetobacter baumannii, Streptococcus sanguis, S. gordonii, and Aggregatibacter actinomycetemcomitans.

As can be seen in Table IV, the Example 7 composition exhibited potent activity against many aerobic and anaerobic bacteria, as well as the fungi.

The MIC of the Example 7 coating composition against S. oralis and S. mitis was noticeably elevated (500 μg/mL) compared to other organisms. It is interesting to note that S. oralis and S. mitis are normal commensals of the oral cavity. Activity of the commonly used antimicrobial chlorhexidine (2% solution) was also determined by the same method. Table IV shows the MIC of the Example 7 coating composition and chlorhexidine (2% solution) as a comparative example against various microorganisms.

Taken together, these results demonstrate that Example 7 possesses potent activity against pathogenic bacteria and fungi commonly isolated from the oral cavity. This activity was more potent than that observed for chlorhexidine.

A similar activity profile was observed for the coating compositions of Examples 10 and 11.

### Example 62

As a further comparison, published data shows that the tested coating composition has a better or at least equivalent MIC compared to CPC alone (i.e. not in a composition according to the barrier coating formulation disclosed herein). See Frank-Albert Pitten and Axel Kramer, “Efficacy of Cetylpyridinium Chloride Used as Oropharyngeal Antiseptic,” Arzneim.-Forsch./Drug Res. 51 (II), pp 588-595 (2001), which is incorporated herein by reference. The data varies based on the microorganism tested, but, for example, CPC (alone) against S. mutans has an MIC of 5.0-6.25 μg/mL, which is much less effective than the 1.95 μg/mL reported in Example 53. This was an unexpected result since CPC has the risk of losing its activity when mixed with other excipient chemicals in a formulation. See Department of Health and Human Services (Food and Drug Administration) (1994) Oral Health Care Drug Products for Over-the-Counter Human Use; Tentative Final Monograph for Oral Antiseptic Drug Products. Proposed Rules (21 CFR Part 356, Docket No. 81N-033A, RIN 0905-AA06). Federal Register 59:6084-124.

### Examples 63-69

### Duration of Antimicrobial Activity of Coating Compositions In Vitro: Determination of Post-Antimicrobial Effect (PAE)

The PAE of Example 8 against several microorganisms was evaluated in Examples 63-68. Control Example 69 was also provided. Several microorganisms were exposed to Example 8 (at a concentration equal to the MIC) for 1 min followed by three washes to remove residual formulation. The treated cells were then spread on agar medium plates, which were incubated at 37° C., and the time taken for the cells to regrow was determined. PAE was expressed as the time (in hours) for which growth inhibition (%) was maintained by the Examples 63-68, compared to the untreated control Example 69.

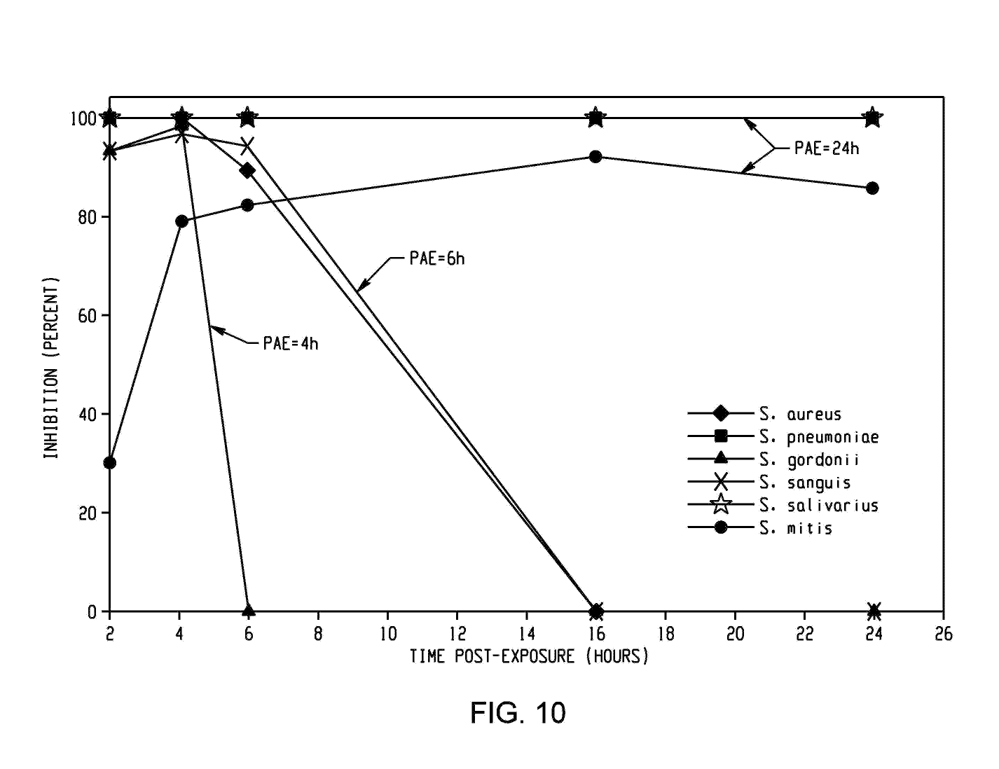

As shown in FIG. 10, Example 8 exhibited a PAE ranging between 4 hours to 24 hours, depending on the organism tested (S. aureus, S. pneumonia, S. gordonii, S. sanguis, S. salivarius, and S. mitis). Similar activity of Example 8 was observed against Candida (data not shown). Other Example coating compositions exhibited similar PAE against microorganisms.

### Example 70

Testing of PAE for the Example 7 coating composition against S. mutans compared to a similar comparative Example with lower CPC content of 0.7% showed that the PAE of Example 7 was 24 hours, while that of Comparative Example 70 was 6 hours. Thus demonstrating that Example 7 exhibits greater prolonged antimicrobial activity than comparative Example 70, and that additional amounts of CPC have more than a simple additive effect on anti-microbial activity.

### Examples 71-76

Scanning electron microscopy was also used to show that treatment of S. sanguis, (Example 71), S. oralis, (Example 72), and C. albicans (Example 73) with the composition of Example 3 resulted in destruction of cellular integrity.

In Examples 71-73, cells were grown in the presence of Example 3 for 24 hours. Next, the cells were washed to remove residual formulation, dehydrated by passing through a series of alcohol solutions (10% to 100%, v/v) and processed for SEM analysis. Control Examples 74-76 differed from Examples 71-73 in that they were not grown in the presence of Example 3.

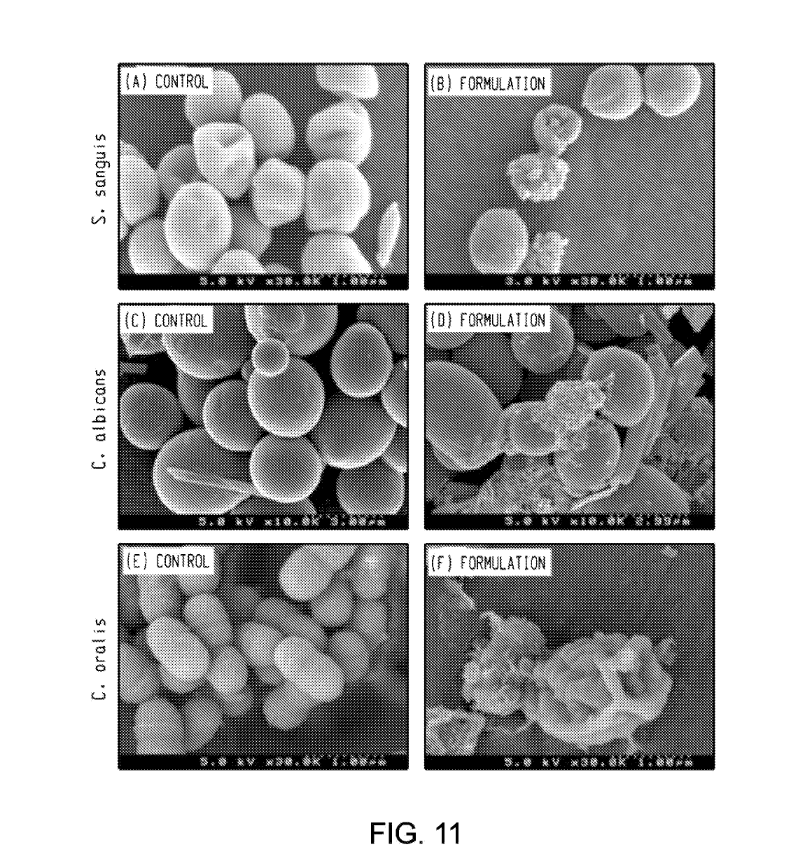

The SEM photos showed that unlike untreated control Examples 74-76, which demonstrated healthy intact cells (FIG. 11 A, C, E), microbes exposed to the Example 3 coating composition were deformed, collapsed, and exhibited total destruction of cellular integrity with clear evidence of leakage of cytoplasmic material. (FIG. 11, B, D, F).

### Examples 77-79

Since biofilms are precursors to certain infectious diseases, in Examples 77-79, experiments were performed to determine whether the coating compositions can prevent formation of biofilms by bacteria and yeasts. Biofilms were formed using an in vitro model. See Chandra et al. “In vitro Growth and Analysis of Candida Biofilms” Nature Protocols 3(12): 1909-1924 (2008).

In Examples 77-79 a standard biofilm model was employed to determine whether the Example 3 coating composition exhibits activity against bacterial and fungal biofilms. In Examples 77-79, three different microorganisms (C. albicans, S. oralis, and S. salivarius) were adhered on substrate for 90 minutes to allow biofilms to form to adhesion phase. Next, discs containing the adherent bacteria were incubated for 15, 30 or 60 minutes with 50% concentration of Example 3 (1:1 dilution with appropriate medium). Following incubation, biofilms were scraped, spread on culture media, incubated and colony forming units (CFUs) were determined Media diluted with phosphate buffered saline (PBS, 1:1) were used as a control. Table V reports data at 0 (Control), 15, 30, and 60 minutes.

TABLE V
Effect of Coating Composition on Early Phase Biofilms (log CFU)

| | Example 77 | Example 78 | Example 79 |
|---|---|---|---|
| Exposure time | C. albicans | S. oralis | S. salivarius |
| Control | 5.44 | 3.25 | 3.16 |
| 15 min | 0 | 0 | 0 |
| 30 min | 0 | 0 | 0 |
| 60 min | 0 | 0 | 0 |

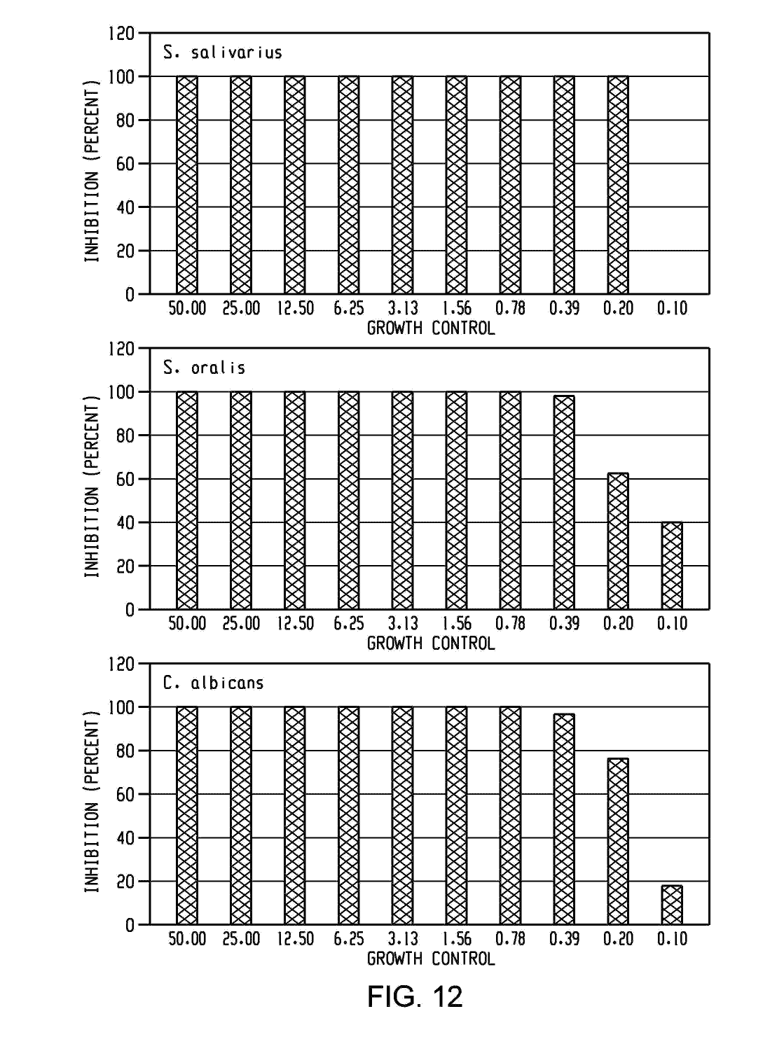

FIG. 12 also reports data on Examples 77-79 as a graph of % inhibition versus growth control. These results showed that Example 3 coating composition inhibited bacterial and fungal microbes with an MIC of 0.2% against biofilms formed by S. salivarius, S. oralis, or C. albicans.

### Examples 80 and 81

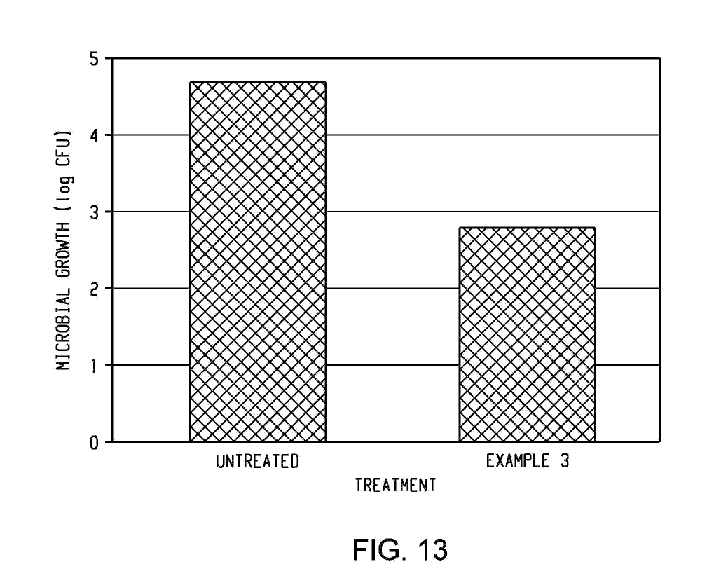

In Example 80 we evaluated the effect of 1 minute exposure of C. albicans early phase biofilms to Example 3, and found that even with an exposure for as short a time as 1 minute, it was able to inhibit biofilm formation (FIG. 13). Example 81 was an untreated control sample.

### Examples 82-84

### Ability of Coating Composition to Treat Mature Biofilms

To determine whether the coating composition can treat biofilms, we evaluated its activity against fully formed mature biofilms. Biofilms were grown to mature phase, and then exposed to Example 7 for 2 or 4 hours, and the resulting CFUs were determined. A composition that causes at least 2-log reduction in microbial CFUs compared to untreated cells is considered to be effective against microbial biofilms.

As shown in Table VI, exposure to Example 7 resulted in complete eradication of biofilms formed by C. albicans and S. oralis, and a 3.4-log reduction in CFUs for biofilms formed by S. salivarius compared to the untreated control (log CFU=3.95 vs. 7.36, respectively).

TABLE VI
Effect of Example 7 on mature biofilms (log CFU)

| | Example 82 | Example 83 | Example 84 |
|---|---|---|---|
| Exposure time | C. albicans | S. oralis | S. salivarius |
| Control | 5.60 | 7.40 | 7.36 |
| 2 h | 0 | 0 | 4.00 |
| 4 h | 0 | 0 | 3.95 |

In summary, the results indicate that Example 7 possesses potent activity against biofilms formed by bacteria and fungi.

### Examples 85-86

### The Coating Composition is Also Active Against Viruses

The activity of coating composition against viruses, including respiratory viruses (influenza virus H1N1, strain 2009/H1N1/infA) and the human immunodeficiency virus (HIV) was determined.

The coating composition inhibits the infectivity of influenza A

To evaluate the effect of the coating composition on the infectivity of influenza virus, Madin Darby canine kidney (MDCK) cells were grown to ≧90% confluence at 37° C. prior to infection. MDCK cells are used routinely for assays involving influenza viruses.

In Example 85 cell monolayers were exposed to the Example 7 coating composition. In control Example 86 the cell layers were exposed to optiMEM (+P/S,+Lglu) tissue culture media for different times: (1) T1: 30 min exposure, (2) T2: 1 h exposure, (3) T3: 2 h exposure. Next, the formulation was removed and the cell monolayers were infected with influenza virus (multiplicity of infection (MOI)=0.1). Cells that were untreated or infected immediately after exposure (T0) were used as baseline controls. Infected cells were then centrifuged, resuspended in 500 μL of growth medium, and incubated at 32.5° C. for 48 hours Immunofluorescence microscopy (using FITC labeled anti-influenza antibody) was also used to evaluate the effect of the Example 7 coating composition on the ability of influenza virus to infect mammalian cells.

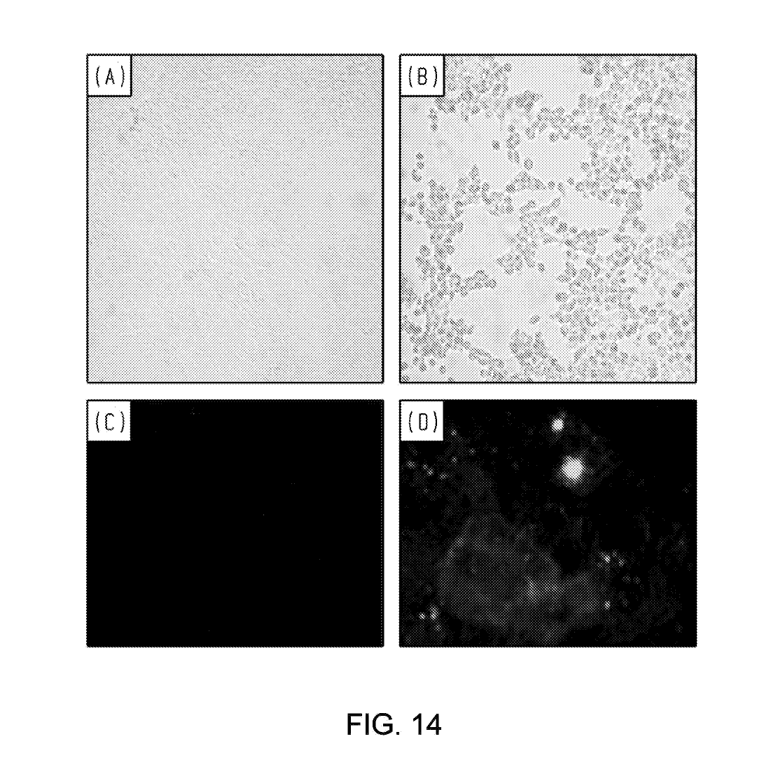

FIG. 14 shows the effect of Example 7 on cytopathic effects of influenza-infected MDCK cells (Example 85) (panels A and C), and control Example 86 (panels B and D). Images were obtained from: phase contrast (A-B), and immunofluorescence microscopy (C-D). No identifying cytopathic effect (CPE) was observed in formulation-treated cells. Untreated cells displayed typical CPE including focal rounding and degenerative changes.

The data showed that exposure of cell monolayers to Example 7 for 30 minutes, 1 hour, or 2 hours remained confluent and healthy (Example 85). In contrast, in the untreated cells and cells treated immediately prior to infection (T0) (control Example 86) demonstrated substantial cytopathic effect. As seen in FIG. 14 panel C, no fluorescence was observed in the coating composition treated cells of Example 85, while the untreated cells of Example 86 exhibited fluorescence (FIG. 14 panel D).

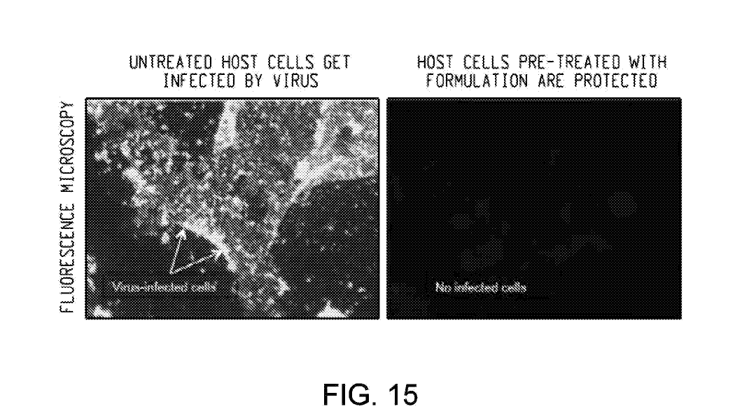

Further fluorescence microscopy images corresponding to Examples 85 and 86 are presented in FIG. 15.

### Examples 87 and 88

### Activity of Coating Composition on Viral Load Using Quantitative PCR

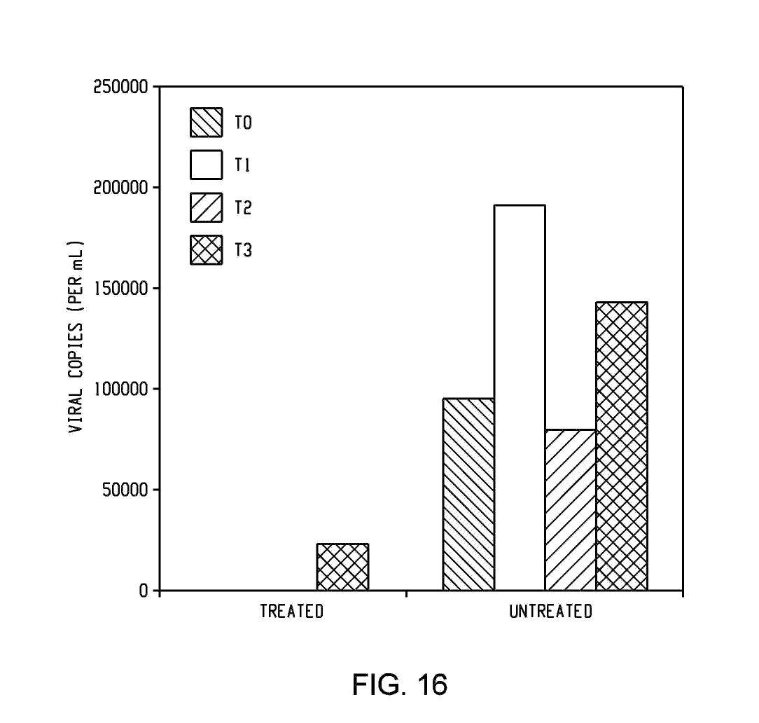

FIG. 16 shows levels of influenza virus in infected treated cells (Example 87) and untreated cells (Example 88), as determined by quantitative PCR. In Example 87, cells were treated with Example 7 and in control Example 88 the cells were left untreated. Later the supernatants were collected and analyzed for the presence of virus.

Cell culture supernatants from the same assay as in Examples 87 and 88 were collected and nucleic acid extracted using QIAamp Viral RNA Kit (QIAGEN, Valencia, Calif.). Random hexamer primers (Invitrogen Carlsbad, Calif.) were used to create a cDNA library for each specimen. Reverse transcription reactions were performed with M-MLV RT (Invitrogen, Carlsbad, Calif.) according to the manufacturer's specifications. Quantitative analysis was performed on a StepOne Plus Taqman Real Time PCR (Applied Biosystems, Branchburg, N.J.) using TaqMan Universal PCR Master Mix (Applied Biosystems, Branchburg, N.J.), 2 μl of cDNA sample, and primers/probes targeting the influenza matrix gene. A reference standard was prepared using a cDNA fragment of the H1N1 matrix gene and human RNAse P amplified by conventional RT-PCR, gel purified (QIAquick, Qiagen, Valencia, Calif.), and quantified using a spectrophotometer (Beckman Coulter, Brea, Calif.).

As shown in FIG. 16 and Table VII, the Example 87 cells treated Example 7 for 30 min or 60 min did not have detectable influenza at 48 hours post infection. Moreover, treatment with Example 7 for 2 hours resulted in a 6-fold decrease in viral load, compared to the untreated control or those treated immediately prior to infection (Example 88).

TABLE VII

| | Example 87 | Example 88 (control) |
|---|---|---|
| 30 min | 0 | 192000 |
| 60 min | 0 | 79800 |
| 120 min | 23400 | 143000 |

### Examples 89-91

### Coating Composition has Direct Antiviral Effect Against Influenza Virus

To determine whether the coating composition has direct antiviral activity against influenza virus, we infected African Green Monkey Kidney (CV-1) cells (grown in 24-well plates to 90% confluence) with influenza virus that was pre-treated with Example 7. CV-1 cells are routinely used a highly susceptible substrate for diagnosis and study of viruses.

In Examples 89-91, a standardized amount of influenza (0.1 MOI) was pretreated for 5 minutes at room temperature with: (1) Example 7 (to form Example 89), (2) control Example 6, a compound without CPC but with preservatives (to form Example 90), and (3) control Example 5 placebo alone (a compound without CPC and preservatives) (to form Example 91). After the 5 minute incubation virus/drug mix was diluted by an additional equal volume with optiMEM (+P/S,+Lglu) to dilute out the treatment compositions.

In Examples 89-91, CV-1 cells were prepared as described in above. The Example 89-91 treated and untreated viruses were then inoculated onto the cells as described above.

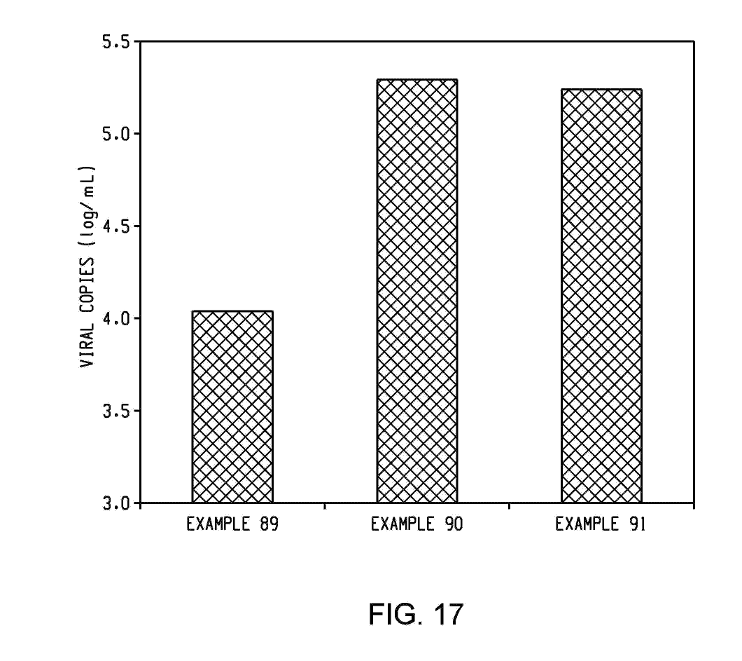

Influenza viral load was determined by real time PCR as described above. The data as shown in FIG. 17 showed significant decrease in viral load for influenza virus pretreated with the Example 7 coating composition containing the antimicrobial agent CPC (Example 89), compared to those containing only the coating composition and/or preservative but no CPC (Examples 90 and 91). Pre-treatment of virus with Example 7 exhibited significant decrease in viral copies, compared to formulations with no CPC.

These results demonstrate that the Example 7 coating composition possesses direct antiviral activity against influenza virus that is not inherent in Examples 5 and 6.

### Examples 92 and 93

In Examples 92 and 93, the coating composition's ability to inhibit the infectivity of influenza A (2009/H1N1/infA) was tested. African Green Monkey Kidney (CV-1) cells were grown in 24-well plates to 90% confluence. Next, the coating composition, Example 7, was applied to the cells (20% Example 7, 80% OptiMeM, working CPC concentration of 0.02%.) in Example 92. Each time point matched with control Example 93 (No coating composition applied, 100% OptiMeM). The coating composition was allowed to dwell on the surface for 30 minutes, and then removed from the ceil monolayer. Cells were thoroughly washed twice with sterile optiMEM (+PfS,+Lglu). Influenza was inoculated at MOi=0.1 at 30 minute intervals from T0 through T+6 hours. Following infection, cells were then centrifuged @ 2200 rpm×30 minutes and 500 μl of optiMEM (+P/S, +Lglu, 2 μg/ml trypsin (sigma-Aldrich, St Louis, Mo.)) was applied. Infected cells were grown at 32.5° C. for 96 hours at 5% CO₂. The influenza viral load was determined by real time PCR.

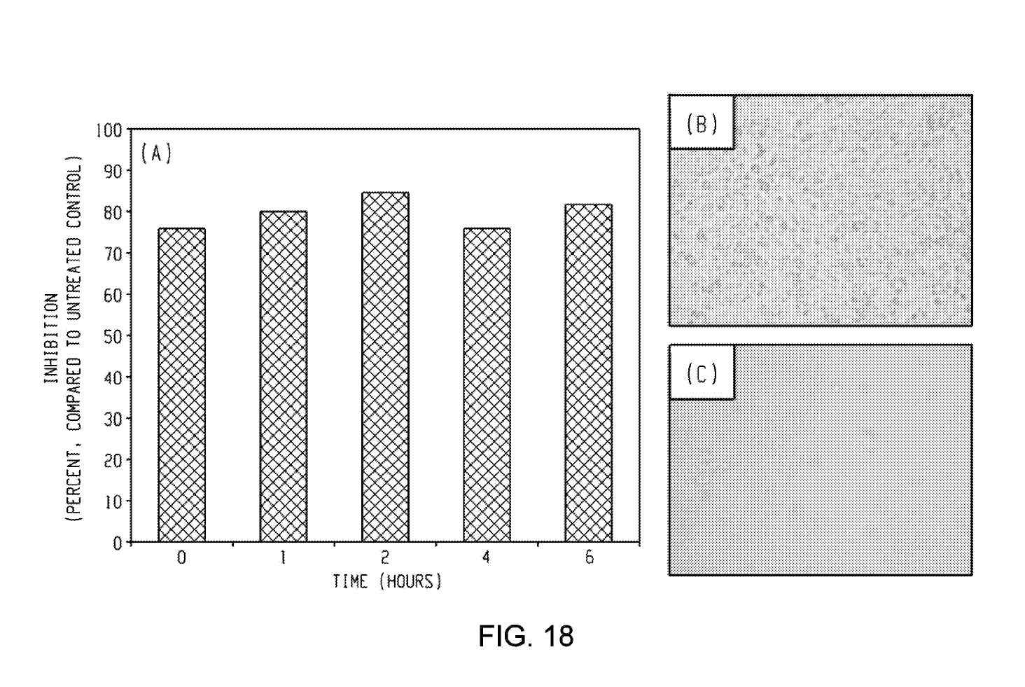

As shown in FIG. 18, pre-treatment of host monolayers with glycerine-xanthan gum formulation results in inhibition of viral infection by up to 84.93% compare to untreated controls. The fact that inhibition of viral infection was observed in host cells despite removal of the coating composition demonstrates that the coating composition formed a protective barrier coating on host cells, which prevented viral invasion for at least 6 hours.

FIG. 1B may be referred to as a possible mechanism accounting for the inhibition of infection.

### Examples 94-96

### Coating Composition Exhibits Activity Against HIV

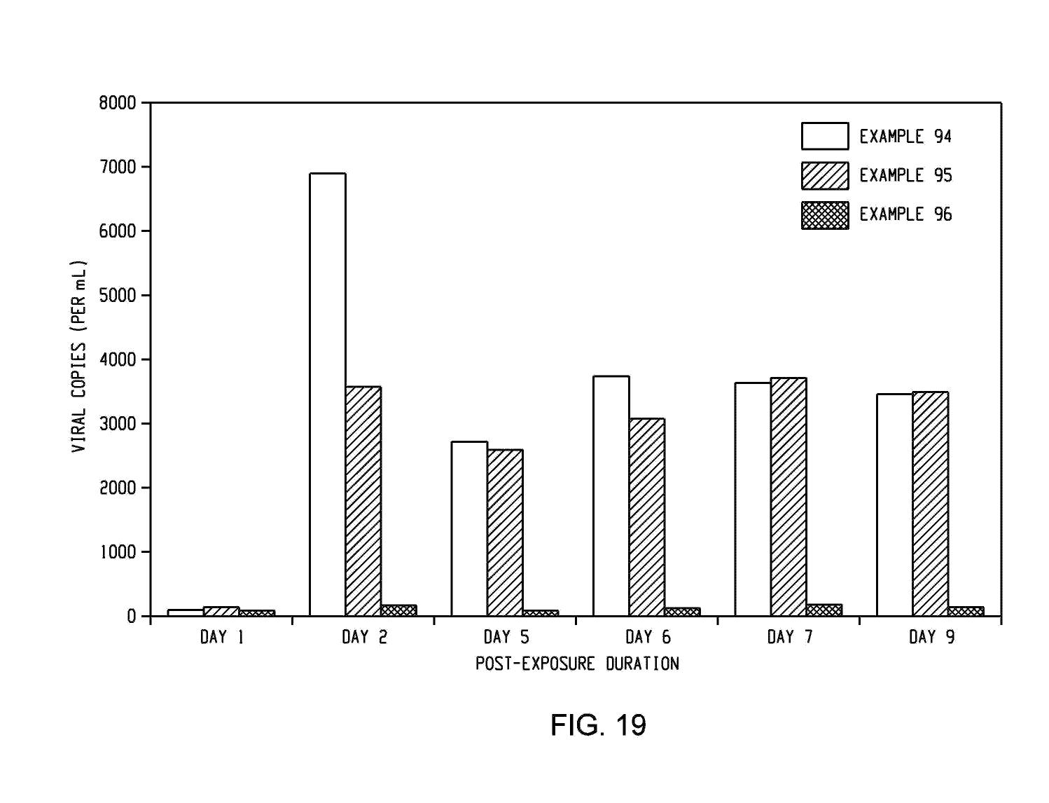

Examples 94-96 determined whether the coating composition possessed activity against HIV. Host MT mammalian cells were plated into 96-well round bottom plates at a density of 15,000 cells/well in RPMI/10% FBS/PS. The next day (Day 2), virus was pretreated with control Example 5 (to form Example 94), control Example 6 (to form Example 95), or Example 7 (to form Example 96) for 5 minutes and added to cells. After 24 hours of exposure to formulation, the MT (macaque) mammalian cells were washed 3 times with phosphate buffered saline (PBS) and fresh media was replaced. Supernatant (10 μL) was collected post-treatment on Days 1, 2, 5, 6, 7, and 9, and the viral load was determined by reverse transcriptase (RT) activity. FIG. 19 shows a graph of the viral copies per mL for each of Examples 72-74 over a 9 day span.

The results showed that Example 7 in Example 96 exhibited anti-HIV activity at all time points monitored post-treatment.

The control Example 5 or control Example 6 without CPC and/or preservative in Examples 94 and 95 exhibited only minimal anti-HIV activity.

In summary, our findings demonstrate that the coating composition Example 7 containing CPC exhibits long-lasting antiviral activity against HIV.

### Example 97

Representative organisms viral lesions are important infections in different mucosal tissues. In Example 97 an experiment was performed to determine whether the coating composition exhibits activity against the common oral Epstein-Barr virus (EBV). Western blotting was used to evaluate the ability of the Example 8 coating composition to degrade lytic viral protein EAD (indicating inhibition of viral replication).

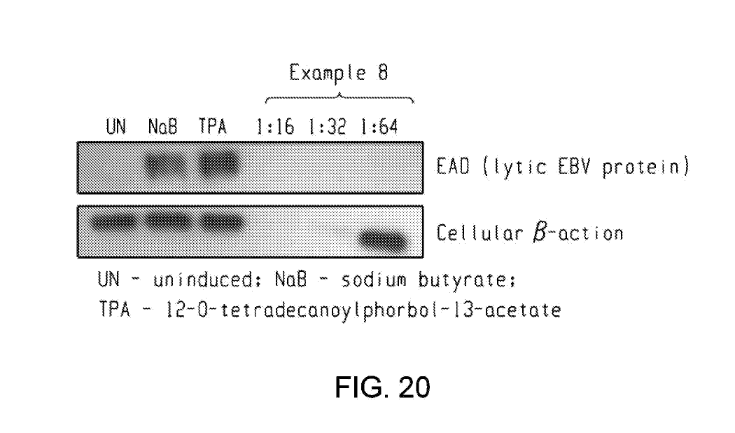

In Examples 97, EBV-infected gastric epithelial cells were exposed to different dilutions (1:16, 1:32 and 1:64) of Example 8, and the presence of EAD protein was detected using specific antibodies. Presence of cellular β-actin was used as an indicator of epithelial cell integrity. As shown in FIG. 20, 1:64 dilution of Example 8 degraded EAD without affecting cellular actin. These results demonstrate that Example 8 specifically inhibits viral replication, and as such, is an effective anti-viral and useful for prevention of viral infection.

### Examples 98-100

### Duration of Anti-Microbial Barrier Versus Commercial Mouthwash Product

To determine the duration for which the coating composition can maintain the antimicrobial activity, bacteria and fungi were exposed to an EHOM of Example 2 that was treated with the coating composition of Example 7 in a well and an EHOM of Example 2 that was treated with a comparative commercial product in a well for 2 minutes. The bacterial and fungal microbes were overlaid on top of the control untreated EHOM (Example 98) and the treated EHOMs (Example 99 and Comparative Example 100). Next the residual (flow-through) solution was removed from the bottom well (lower chamber of the EHOM model) and spread onto agar medium plates. FIG. 3 depicts this test method for further clarity. These plates were then incubated at 37° C., and the number of microbial cells (colony forming units, CFUs) growing after 24 hours were counted.

In control Example 98 an untreated EHOM was tested. In Example 99 S. mitis bacteria was overlaid on the coating composition as described above. Example 100 is a comparative example showing the activity of commercially available LISTERINE (containing ethanol (26.9%), menthol, thymol, methyl salicylate, and eucalyptol) against S. mitis bacteria. Table VIII shows the results.

TABLE VIII

| | CFUs of S. mitis bacteria in flow | | |
|---|---|---|---|
| | through liquid from EHOM | | |
| Time post- | Example 98 | | Example 100 |
| exposure | (control) | Example 99 | (comparative) |
| 2 hours | 1150000 | 5820 | 780000 |
| 4 hours | 1400000 | 5500 | 800000 |
| 6 hours | 1600000 | 6000 | 840000 |

### Examples 101-103

In Examples 101-103, the same procedure of Examples 98-100 was performed except Candida albicans fungus was tested on the coating composition as described above. Table IX shows the results. Example 103 is comparative, showing the activity of commercially available LISTERINE.

TABLE IX

| | CFUs of Candida albicans in flow | | |
|---|---|---|---|
| | through liquid from EHOM | | |
| Time post- | Example 101 | | Example 103 |
| exposure | (control) | Example 102 | (comparative) |
| 2 hours | 1150000 | 12000 | 124000 |
| 4 hours | 2900000 | 12000 | 252000 |
| 6 hours | 3900000 | 13000 | 350000 |

The data further showed that Example 7 coating composition maintained activity for up to and including 24 hours. Taken together, these results showed that unlike LISTERINE, the Example 7 coating composition continued to maintain an intact barrier on EHOM tissues for up to and including 24 hours.

### Examples 104-153

Examples 104-153 were performed to identify further examples of concentrations of glycerin and xanthan gum that can form a barrier effective in preventing the passage of microorganisms. Since this application does not require a barrier that prevents passage of microorganisms, this data is omitted. However, patent publication U.S. 2012/0270909 as well as the provisional applications that this application claims the benefit of priority to, include this information.

It should be noted that an effective barrier coating for a surface treatment may be formed at lower concentrations of glycerine and/or xanthan gum when an effective antimicrobial is added. This is because the antimicrobial and barrier coating act in tandem to stop and/or kill the harmful microbes. In the case of a composition for applying to an inanimate surface, it is not so important to block passage of a microorganism to the other side of the barrier coating, since an inanimate surface cannot be infected.

### Examples 154-160

Examples 154-160 were performed to demonstrate safety of the composition on mucosal surfaces. Patent publication U.S. 2012/0270909 as well as the provisional applications that this application claims the benefit of priority to include this information.

### Example 161

### Glycerine-Xanthan Gum Formulations Form a Coating on the Human Oral Mucosa

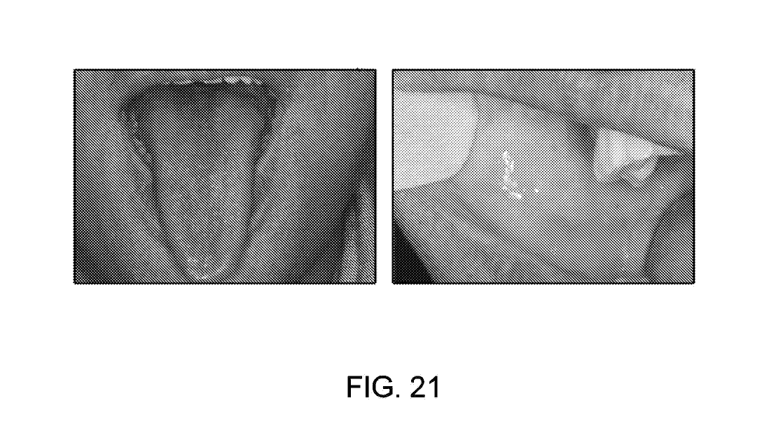

To determine whether glycerine-xanthan gum formulation can form a coating on the human oral mucosa, we spiked the Example 7 formulation with Gentian Violet (GV) as a marker dye. The spiked product (750 μL) was sprayed onto the oral cavity of human volunteers. Post-application, the oral cavity was inspected for staining, and the images were captured using a digital camera. As shown in FIG. 21, the formulation stained both cheeks and the dorsal/ventral surface of the tongue.

### Examples 162 and 163

### Exposure of Microbes to Coating Composition Inhibits Cell Growth: Time-Lapse Microscopy

To determine the inhibitory activity and duration for which coating compositions exhibit activity against microbes, time-lapse analysis was performed on cells exposed to the coating composition, compared to untreated bacteria and fungi.

In Example 162, S. mutans microbial cells were exposed to Example 7 for one minute, washed to remove any residual agent, and allowed to grow in a petri-dish containing fresh growth medium. Growth of organisms at 37° C. was monitored for a 6 hour period, and photomicrographs were taken every 20 minutes over the 6 hour incubation period using a camera connected to the microscope.

In control Example 163 the same procedure was followed with untreated cells.

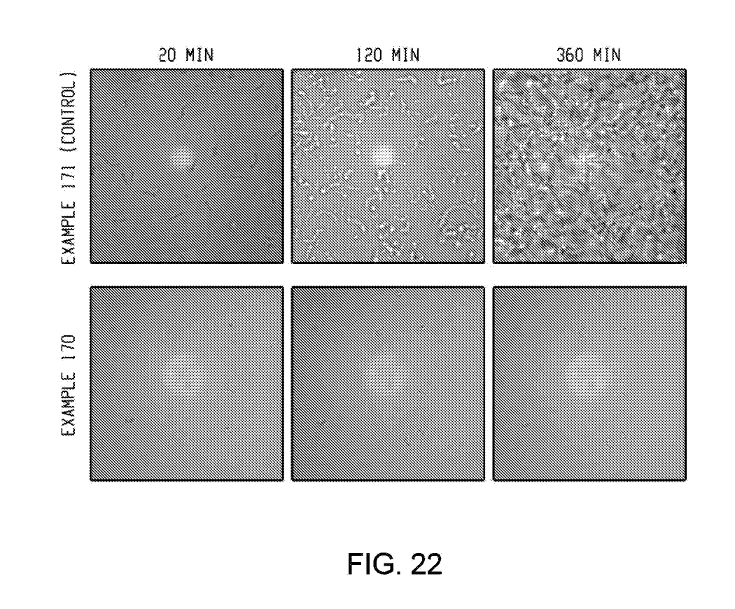

As shown in FIG. 22, in contrast to the untreated bacteria, where cells reached confluence by 6 hours, microbes treated with the Example 7 coating composition failed to regrow during the same time period post-exposure. Similarly, exposure of Candida cells to the Example 7 coating composition completely inhibited growth during the incubation period (data not shown).

These results further confirmed that the coating composition possesses prolonged antimicrobial activity.

### Examples 164-166

### In vivo Study

### Coating Composition (Example 7) Lowers the Oral Microbial Load in Humans: Short- and Long-Term Activity

Short-Term Activity

The duration of activity of Example 7 was determined in healthy individuals by evaluating the effect of a single application on microbial burden of the oral cavity. In Examples 164-166, three healthy individuals (over 18 years of age, healthy mouth) were enrolled with informed consent, and asked to apply a single application of the composition of Example 7 on their cheeks. A single application was defined as three sprays of 0.25 ml each in volume. Next, swabs were collected from these individuals at baseline (pre-treatment), 1 hour, 2 hours, and 6 hours post-treatment. Swabs were cultured on agar media plates specific for aerobic or anaerobic organisms, incubated for 24-28 hours at 37° C., and the number of CFUs were counted. Effect of Example 7 on microbial burden was determined (CFUs), and percentage inhibition was calculated for each post-exposure time point relative to the baseline (0 minutes) CFUs.

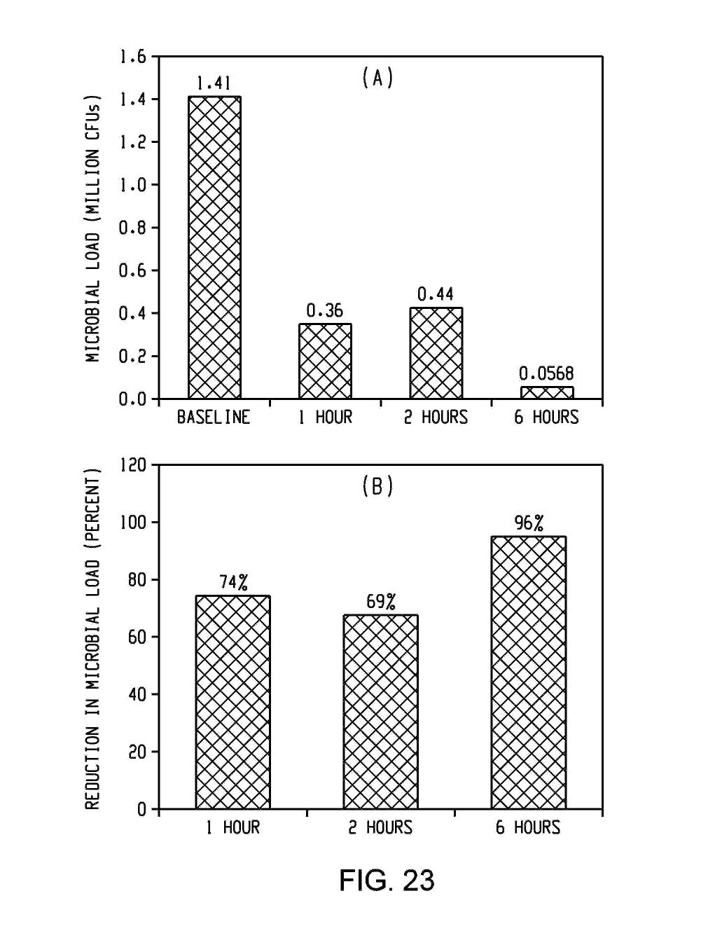

The results showed that application of Example 7 led to consistent reduction in microbial load for up to 6 hours (See FIG. 23A, which shows CFUs of a representative tested individual. Treatment with the coating composition resulted in 69% to 96% reduction of the microbial burden in the oral cavity (See FIG. 23B, which shows a representative individual's reduction in microbial load.)

### Examples 167-169

### Long-Term Activity

The activity of the coating composition over a 5-day period against oral microbes was evaluated. In Examples 167-169, three healthy individuals were enrolled, and asked to apply a single dosage (three sprays 0.75 mLs total) of Example 7 three times daily (approximately 9 AM, noon, and 3 PM) for a 5-day period (representing a typical 5-day work-week). Swabs were collected from these individuals at baseline (before application on day 1) and at the end of the day on each day during the 5-day period. Collected swabs were cultured on agar media plates, incubated for 24-28 hours at 37° C. and at 5% CO₂ humidity, and the number of CFUs were counted.

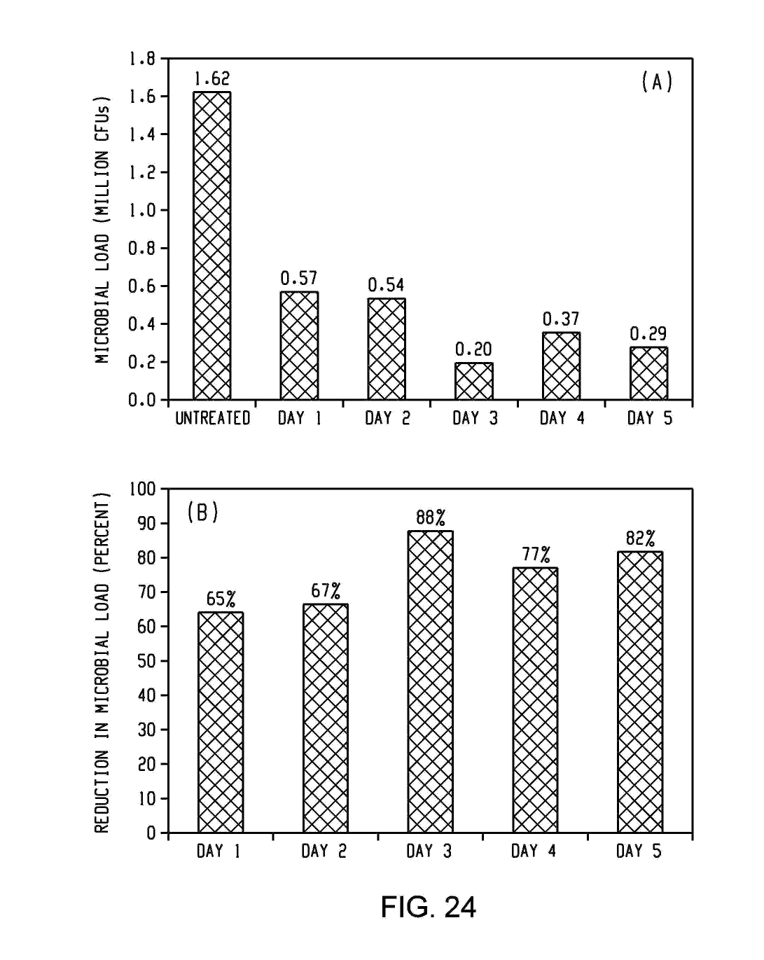

The effect of the Example 7 coating composition on microbial burden was determined (as median CFUs for the three subjects), and percentage inhibition was calculated for each post-exposure time point relative to the baseline (0 min) CFUs. FIG. 24 shows these results in a graph of CFUs versus time (FIG. 24A) and reduction in microbial load versus time (FIG. 24B). Examples 167-169 demonstrate that application of Example 7 over 5 days led to consistent reduction in microbial load over the 5-day test period (FIG. 24A). Treatment with the Example 7 coating composition resulted in 65%-88% reduction of the median microbial burden in the oral cavity of the study participants (FIG. 24B).

### Examples 170-198

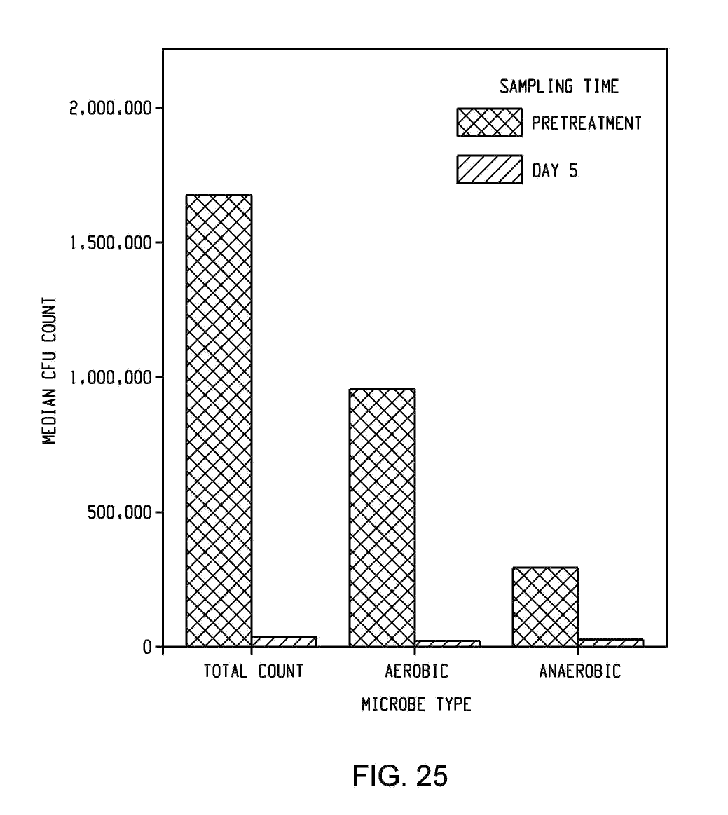

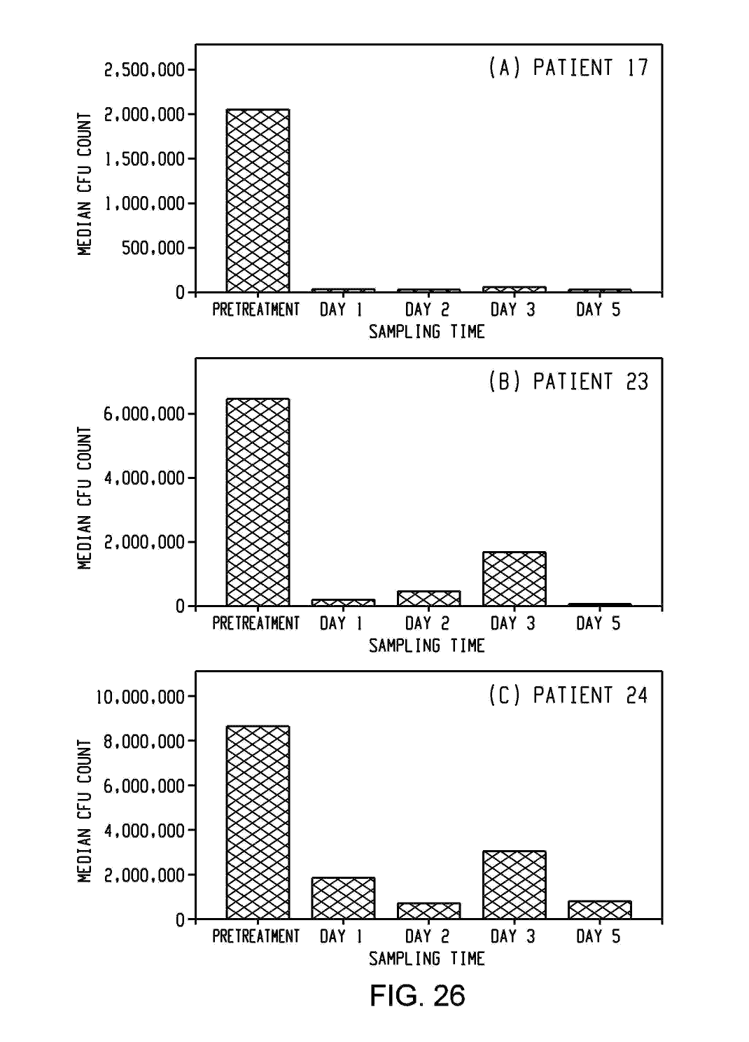

In a clinical study, twenty-nine healthy individuals were enrolled after informed consent. Baseline information was recorded (age in years, gender, ethnicity, and date of enrolment). Oral examination of the mouth was undertaken, and the inside of the mouth (cheek) was swabbed with a sterile culture swab. Baseline oral swab samples were cultured to determine bacterial load prior to study. In Examples 170-198, each of the twenty-nine participants were given a spray bottle containing the coating composition of Example 7 and instructed to spray the inside of their mouth for a total volume of 0.75 ml, then swish for 30 seconds and swallow. Two groups of approximately equal number of participants were tested. One group used the example coating composition every two hours, three times a day, for five days (a typical work week). The other group used the example coating composition every two hours, four times a day, for five days (a typical work week). No substantial difference was noted in the two groups. Swabs were collected on days 1, 2, 3, and 5 at the end of the day (8 hours after the first administration of the coating composition) and cultured on media specific for aerobic and anaerobic bacteria. Data were presented as number of microbes: total, aerobic and anaerobic. FIG. 25 shows a graph of total microbial load and breaks down the total into aerobic and anaerobic counts from just prior to treatment and on day 5 of treatment. FIG. 26 shows graphs of microbial load over the 5 day period in oral samples obtained from three representative study participants.

Overall, the in vivo testing showed that the coating composition exhibits antimicrobial activity against oral microbes, as measured by reduction in the levels of these organisms, over both short- and long-term duration.

The data showed that treatment with the coating composition over a 5-day period resulted in reduction in the oral microbial load, for total microbes, aerobic and anaerobic organisms.

### Example 199-205

### Identification of Additional Humectants for Forming a Barrier to Prevent Microbial Penetration

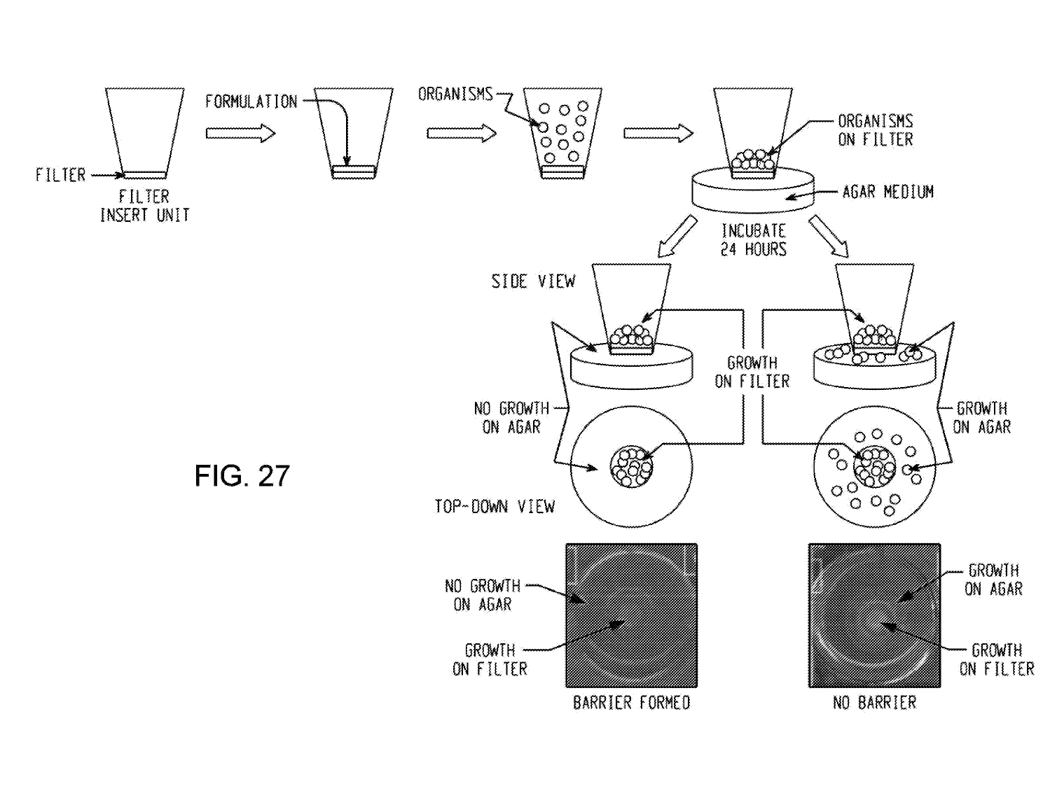

In Example 199 an in vitro filter insert-based model (see FIG. 27) was used to test different humectants at different concentrations.

Six compositions were prepared according to Table X based on the mixing procedures used for Examples 3-8.

TABLE X

| | Ex. | Ex. | Ex. | Ex. | Ex. | Ex. | Ex. |
|---|---|---|---|---|---|---|---|
| | 199 | 200 | 201 | 202 | 203 | 204 | 205 |
| Xanthan | 0.4 | 0.4 | 0.4 | 0.4 | 0.4 | 0.4 | |
| Gum | | | | | | | |
| Glycerin | 4.5 | 4.5 | 4.5 | | | | 4.5 |
| Sorbitol | | 4.5 | 4.5 | 4.5 | | 4.5 | |
| Xylitol | | | 4.5 | 4.5 | 4.5 | | 4.5 |

Next, 100 μL of Examples 199-205 were placed into filter inserts (pore size 0.8 μm diameter, that allows both bacteria and fungi to pass through) and allowed to form a layer. Next, organisms were overlaid on the layer formed by the test solutions. The filter inserts containing the layer of test solutions and microorganisms were then placed on the surface of agar media plates and incubated for 24 hours at 37° C. After the incubation period, the agar media plates were evaluated for growth on filter insert and in the agar media. Growth on filter insert but no growth in agar media indicated that the test solution formed a barrier, which prevented the microbes from passing through. In contrast, microbial growth in the filter insert as well as the agar media indicated that no such barrier was formed.

The results showed that each of the xanthan gum-based solutions containing the tested humectants (singly or in combination) formed intact barriers on the filter insert that prevented the passage of microorganisms into underlying agar medium.

### Example 206-213

### Determination of the Solubility Limits of Xanthan Gum

To determine the solubility of xanthan gum, it was mixed at different concentrations in water and the solubility observed by monitoring the presence or absence of clumps and free flow of the mixture. Table XI reports the results and concentrations.

TABLE XI

| | Xanthan Gum | |
|---|---|---|
| Example | Concentration | Solubility |
| 206 | 0.40% | free flowing viscous solution |
| 207 | 0.45% | some clumps, viscous solution |
| 208 | 0.5% | more clumps, viscous solution |
| 209 | 0.6% | clumps, more viscous than above |
| 210 | 0.7% | clumps, more viscous than above |
| 211 | 0.8% | Extensive clumps, highly viscous solution, |
| | | no free flow |
| 212 | 0.9% | Extensive clumps, highly viscous solution, |
| | | no free flow |
| 213 | 1.00% | Extensive clumps, highly viscous jelly, |
| | | no free flow |

We found that when mixed at 0.4%, xanthan gum formed a free-flowing viscous solution (Table XI). In contrast, mixtures containing 0.45% or 0.5% xanthan gum formed a viscous fluid but contained small clumps. The extent of clumps increased with increasing concentration of xanthan gum (0.6% and 0.7%). At concentrations ≧0.8%, xanthan gum mixture contained extensive clumps, with a jelly-like consistency and no free flow.

### Example 214

### Comparison of Cationic CPC in Coating Composition with Neutral Antimicrobial Agent in Coating Composition

In Example 214, the formulation of Example 7 was made, except the neutral agent Citral was used instead of CPC. The antimicrobial activity of formulations containing CPC (0.1%) or Citral (0.5%) against Streptococcus was ascertained. The assay described above in Examples 48-61 was used to perform these studies.

The results showed that the formulation containing citral exhibited antimicrobial activity (MIC=12.5%). However, activity of formulation containing citral was significantly less potent than that containing CPC (MIC=0.098%).

### Example 215

### Physico-Chemical Testing of Hydrophobicity and Comparison

In Example 215 thin layer chromatography analysis was used to compare the hydrophobicity of Example 7 with a hydrophobic composition. The hydrophobic composition was comprised of the components in Table XII.

TABLE XII

| | Wt % |
|---|---|
| Glycerin | 7 |
| Sorbitol | 5 |
| Poloxamer 338 | 1 |
| PEG 60 Hydrogenated castor oil | 1 |
| VP/VA copolymer | 0.75 |
| Sodium benzoate | 0.5 |
| Cellulose Gum | 0.2 |
| CPC | 0.05 |
| Methyl Paraben | 0.05 |
| Propyl paraben | 0.05 |
| Sodium Saccharin | 0.05 |
| Xanthan Gum | 0.01 |
| Disodium Phosphate | 0.006 |
| Flavoring and coloring agents | 0.121 |

*the remainder of the composition was purified water

10 μL of Example 7 and the hydrophobic composition were deposited on pre-made TLC plates (at a distance of 2 cm from the bottom edge). The spots were air-dried for 5 minutes, and the plates were placed in a TLC chromatography jar containing water as a solvent. The TLC system was allowed to run until the solvent front reached the top edge of the plate. Plates were removed and the solvent and sample fronts were marked. The Relative Front (Rf) values were calculated for the two samples using the formula I:

I. Rf=Distance Travelled by Spot/Distance Travelled by Solvent Front

The results showed that the Rf value for the hydrophobic composition and Example 7 were 0.33 and 0, respectively, indicating that the hydrophobic composition was highly miscible in water. In contrast, Example 7 did not exhibit any mobility in the aqueous solvent, demonstrating that this formulation is hydrophobic or not hydrophilic.

### Example 216

A coating composition was made by mixing the components according to Table XIII below in water to form a solution. A eucalyptol component was also included in an amount of 5× per the Homeopathic Pharmacopeia. This did not affect the tested results, but it demonstrated that the composition still works as a durable antimicrobial with this component added into it. All percentages are by weight.

TABLE XIII

| | | Humectant | |
|---|---|---|---|
| | Antimicrobial (CPC) | (Glycerin) | Gum (Xanthan Gum) |
| Example 216 | 0.01% | 35% | 0.4% |

### Examples 217-219

The coating composition was also shown to have effectiveness in killing allergy causing molds. MIC tests were performed on a polystyrene plastic surface.

In Example 217 the coating composition of Example 216 was tested to determine its MIC against Stachybotrys MRL 9740. The Example 7 composition had an MIC of 0.06 micrograms/ml.

In Example 218 the coating composition of Example 216 was tested to determine its MIC against Aspergillus fumigatus 18748. The Example 7 composition had an MIC of 0.49 micrograms/ml.

In Example 219 the coating composition of Example 216 was tested to determine its MIC against Cladosporium. The Example 7 composition had an MIC of 0.39 micrograms/ml.

Because Stachybotrys and Aspergillus fumigatus are mold-causing organisms, these examples further support the embodiment wherein the coating composition is applied to surfaces to prevent or treat mold growth or discoloration.

### Examples 219-224

In Examples 219-224 the effect of coating composition on MRSA biofilm formation on a silicone elastomer disc surface was evaluated.

In Examples 219-221, three silicone elastomer discs with a 1 cm diameter were pre-sprayed with 0.25 mL with the Example 7 coating composition for 60 min and incubated at 37° C. In Examples 222-224 a control example was performed by treating a silicone elastomer disc with an equivalent amount of a phosphate-buffered saline (PBS) for 60 minutes and incubated at 37° C.

The Example 219-224 pretreated discs were each submerged in 4 mL MRSA suspension (1×10⁷ cells/mL), and incubated at 37° C. for 90 min (“Adhesion Phase”). Next, the discs with adherent cells were removed and transferred to wells containing 4 mL of Brain Heart Infusion (BHI). The wells were incubated at 37° C. on a rocker for 24 hours. Biofilm formation on the discs was evaluated by quantitative culturing on BHI agar plates. Scanned images of the wells were recorded using a scanner.

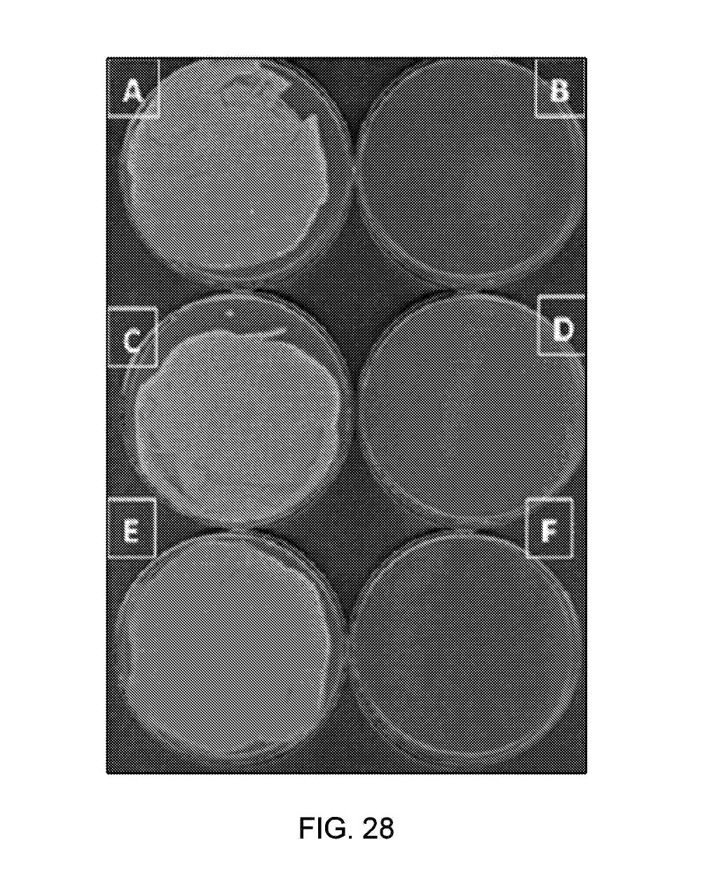

As shown in Table XIV, pre-treatment with Example 7 coating composition prevented formation of biofilms on the disc surface. FIG. 28 shows images of colony burden in biofilms formed by MRSA on the PBS treated (A, C, E) and Example 7 treated (B, D, F) discs.

TABLE XIV

| Treatment | Example | MRSA CFUs/mL |
|---|---|---|
| Example 7 | 219 | 0 |
| coating | 220 | 0 |
| composition | 221 | 0 |
| PBS | 222 | 1.58 × 10⁸ |
| | 223 | 1.72 × 10⁸ |
| | 224 | 1.53 × 10⁸ |

### Examples 225-246

The Example 7 coating composition was tested to determine its efficacy against several strains of Bordetella pertussis. In test Examples 225-235, agar-based assays were constructed in which Example 7 coating composition was incorporated in Regan-Lowe Charcoal agar BBL #297883 plates as a 64 microgram/mL dilution in water. Control Examples 236-246 were agar plates containing no Example 7 coating composition. In each of Examples 225-246 5×10⁴ cells (50 uL) of Bordetella pertussis were spotted on the test surface and plates were incubated at 37 degrees C. for 24 hours. As shown in Table XV, confluent growth was observed in control Examples 236 to 246, while no growth was observed in test Examples 236-246. The designation 4+ means luxurious growth.

TABLE XV

| | Bordetella | Microbial |
|---|---|---|
| Example | pertussis Strain # | Growth |
| 225 | J11E | None |
| 226 | J11F | None |
| 227 | J14B | None |
| 228 | J14C | None |
| 229 | J14D | None |
| 230 | J14G | None |
| 231 | J32B | None |
| 232 | J32C | None |
| 233 | J32D | None |
| 234 | J36E | None |
| 235 | J36F | None |
| 236 | J11E | 4+ |
| 237 | J11F | 4+ |
| 238 | J14B | 4+ |
| 239 | J14C | 4+ |
| 240 | J14D | 4+ |
| 241 | J14G | 4+ |
| 242 | J32B | 4+ |
| 243 | J32C | 4+ |
| 244 | J32D | 4+ |
| 245 | J36E | 4+ |
| 246 | J36F | 4+ |

### Examples 247-252

The antiviral activity of the coating composition, Example 7 (in various diluted concentrations) was evaluated against the ATCC VR-1200 strain of rhinovirus.

Human Hepatoma (HUH-7) Cells were prepared in 24-well plates with Dulbecco's Modified Eagle Medium (DMEM) with 10% heat inactivated fetal calf serum and supplemented with L-glutamine (Lglu) and penicillin/streptomycin (P/S) (unless specified, all reagents produced by Gibco, N.Y., USA). All culture cells were grown to 90-100% confluence at 37° at 5% CO₂ and then washed with OptiMEM +P/S +Lglu once prior to infection.

In Examples 247-251, the Example 7 composition was applied to cell monolayers at varying concentrations (5%, 10%, 15%, 20%, 50% diluted in 400 microliter optiMEM (+P/S, +Lglu)) for a working CPC concentration of 0.005%, 0.01%, 0.015%, 0.02% and 0.05% respectively, and was allowed to dwell for 1 hour prior to inoculation. In control Example 252 400 microliter optiMEM (+P/S,+Lglu) was applied to the cells and allowed to dwell for 1 hour prior to inoculation.

The cell monolayers were then removed from the Example 7 dilutions or control optiMEM and rhinovirus was applied at a multiplicity of infection (MOI) of 0.1. Cells were incubated with virus at 32.5° C. for 1 hour. After which the inoculum was removed and 500 ul OptiMEM+P/S+Lglu was placed on the cells. Cells were then grown at 32.5° C. at 5% CO2. After 5 days incubation, cell culture supernatants were collected for rhinovirus viral load quantification.

Rhinovirus viral titer of the Example 247-251 cell culture supernatants were measured by real time PCR. In comparison to Control Example 252 significantly decreased rhinovirus viral load was demonstrated in Example 251, which was a 50% concentration of Example 7. See Table XVI below.

TABLE XVI

| Example | Wt. % Example 7 | Amount | Viral load/mL |
|---|---|---|---|
| 247 | 5% | 303354.64 | 12141854.69 |
| 248 | 10% | 5628.209 | 2251283.75 |
| 249 | 15% | 92717.83 | 37087131.25 |
| 250 | 20% | 8776.60 | 3510638.67 |
| 251 | 50% | 0 | 0 |
| 252 | 0 (control) | 95307.36 | 38122943.75 |

### Examples 253 and 254

A test Example 253 was formulated with a 50% Example 7 diluted suspension (0.05 CPC concentration) in 500 microliter optiMEM (+P/S,+Lglu). A control Example 254 was formulated as a control solution with no Example 7 (500 microliter optiMEM (+P/S,+Lglu)). Examples 253 and 254 were applied the cells disclosed in Examples 246-252, but at defined intervals: T-1 hour, T-30 min, and T-0 (Immediate) prior to infection.

The cell monolayers were then removed from the Example 253 suspension and the Example 254 control solution. The rhinovirus viral particles were applied to the treated cell monolayers at a multiplicity of infection (MOI) of 0.1. Cells were incubated with virus at 32.5° C. for 1 hour. After which the inoculum was removed and 500 ul OptiMEM+P/S+Lglu was placed on the cells. Cells were then grown at 32.5° C. at 5% CO₂ for 5 days. The cells treated with Example 253 and 254 were viewed daily for the presence of cytopathic effect. After 5 days incubation, cell culture supernatant was gathered for immunofluorescence and rhinovirus viral load quantification.

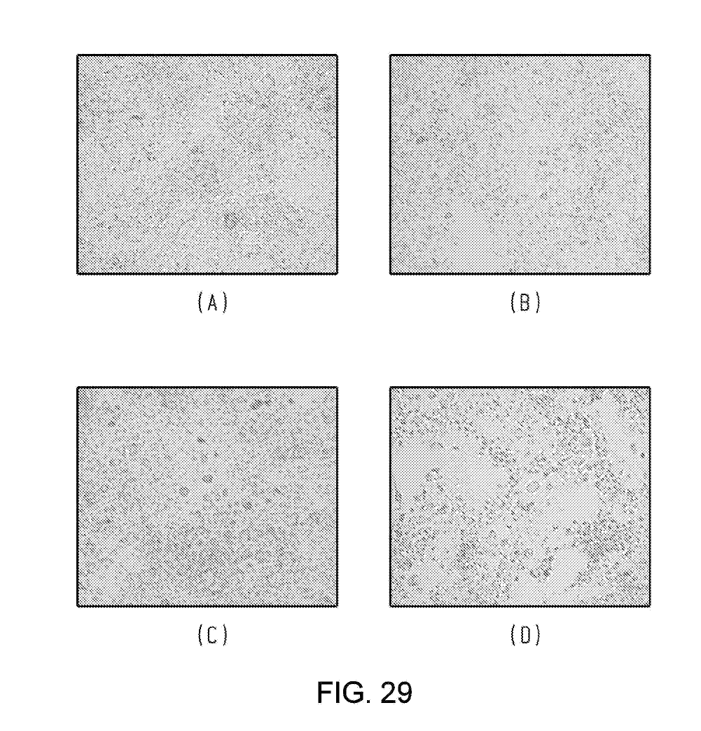

FIG. 29 discloses photos of cells treated with test Example 253 at FIG. 29(a) T—1 hr, FIG. 29(c) T—30 min and FIG. 29(b) T 0 (immediate). None of these photos demonstrated any cytopathic effect and healthy cells overgrew the plate. However, as shown in FIG. 29(d) the Example 254 untreated control cells demonstrated focal rounding, detachment and cell death. Cytopathic effect determination included the development of focal rounding, cell size enlargement or reduction, syncytial formation, development of multinucleated giant cells, and detachment.

Immunofluorescence was determined as follows: Virus infected cell monolayers and uninfected control were washed with sterile PBS. The cells were trypsinized, spotted upon wells on slides and fixed with acetone. The slides were tested by DFA employing FITC labeled monoclonal antibodies. An indirect immunofluorescence assay was performed using Light Diagnostics Pan-Enterovirus Detection Kit (Millipore). This detection kit is well described for having cross reactivity with rhinovirus infected cells. All antibody dwell steps occurred for 1 hour at 37° C. Following a final wash, cells were evaluated at a wavelength of 488 nm for the presence of fluorescence.

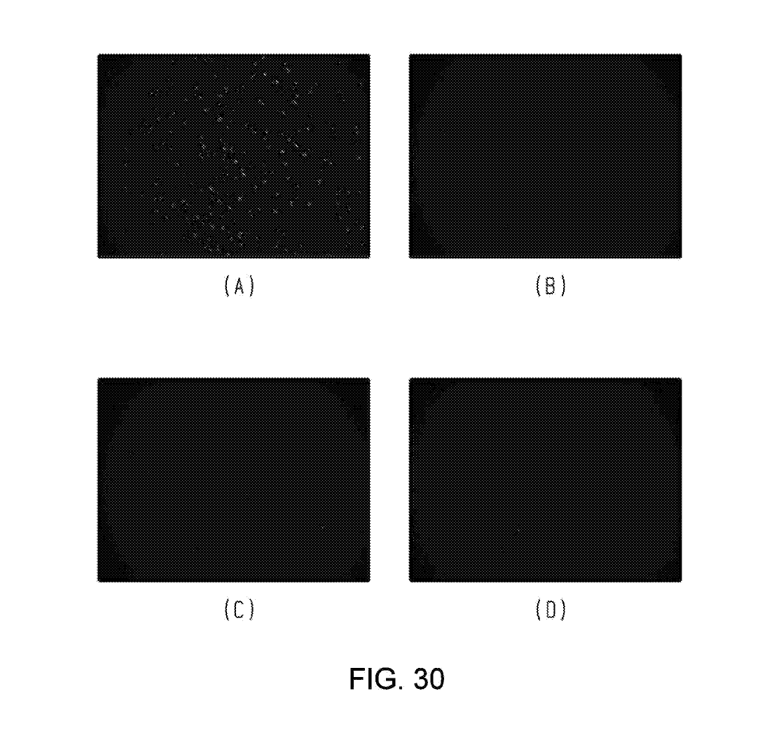

FIG. 30 discloses immunofluorescence photos of cells pretreated treated with test Example 253 at FIG. 30(d) T—1 hr, FIG. 30(b) T—30 min and FIG. 30(c) T-0 (immediate). The cells treated with Example 253 for 1 hour and 30 minutes displayed no immunofluorescence. The cells treated with Example 253 for T-0 (immediate) demonstrated scant fluorescence. However, the untreated control Example 254 showed substantial immunofluorescence suggesting profound viral infection (FIG. 30(a)).

Viral load for the samples was quantified as follows: Cell culture supernatants were collected and stored at −80° C. Nucleic acid was extracted using QIAamp Viral RNA Kit (QIAGEN, Valencia, Calif.). Random hexamer primers (Invitrogen Carlsbad, Calif.) were used to create a cDNA library for each specimen. Reverse transcription reactions were performed with M-MLV RT (Invitrogen, Carlsbad, Calif.) according to the manufacturer's specifications. Quantitative analysis was performed on a StepOne Plus Taqman Real Time PCR (Applied Biosystems, Branchburg, N.J.) using TaqMan Universal PCR Master Mix (Applied Biosystems, Branchburg, N.J.), 2 microliter of cDNA sample, and primers/probes targeting the rhinovirus polyprotein gene. A reference standard was prepared using an amplicon amplified by conventional RT-PCR, gel purified (QIAquick, Qiagen, Valencia, Calif.), and quantified using a spectrophotometer (Beckman Coulter, Brea, Calif.). The results are shown in Table XVII.

TABLE XVII

| | Amount | Viral load/mL |
|---|---|---|
| Example 253: | 0 | 0 |
| 1 hour pretreatment | | |
| Example 253: | 0 | 0 |
| 30 minute pretreatment | | |
| Example 253: | 0 | 0 |
| Immediate pretreatment | | |
| Example 254 (control) | 331025.2 | 1.32 × 10⁸ |

No rhinovirus amplification was apparent at T-1 hour, T-30 min, or T-0 (immediate) timepoints at 5 day post infection. Untreated (control) cells demonstrated substantial amplification (>10⁸ copies/ml) suggesting viral infection.

### Example 255

### Cetylpyridinium Chloride Composition Exhibits Antimicrobial Activity on Inanimate Surfaces

In Example 255 a cetylpyridinium chloride-based spray disinfectant was evaluated for its activity against methicillin-resistant Staphylococcus aureus (MRSA). The antibacterial effect of pre-coating surfaces with the composition was analyzed, and the effect of a water rinse on maintaining its activity was also analyzed.

In an embodiment, the coating composition containing cetylpyridinium chloride retains a substantial amount of its cidal or static activity even on stainless steel surfaces after washing with water, such as at least about 35%, about 35% to about 50%, or about 15% to about 40% of cidal or static activity after washing with water.

The test CPC composition had the following formula: 93% to 97% water, 0.5 to 1% CPC antimicrobial, 0.5 to 1% glycerin, with the remainder of the composition comprising preservatives, such as cremophor RH 60, copovidone, parabens, and sodium benzoate, none of which were present in an amount more than about 1%.

The activity of the test CPC composition (CPC_{sd}) was evaluated by soaking stainless steel carriers with MRSA suspension (1×10⁸ cells) for 15 min at 37° C. Next, excess fluid was drained, the carriers sprayed with CPC_{sd} (0.5 mL dosages) for 30 seconds, air dried, and incubated in Brain heart infusion medium (BHI) overnight. Aliquots of the medium were then quantitatively cultured.

To determine the effect on pre-coated carriers, discs were sprayed with CPC_{sd}, (0.50 mL dosages), air-dried for 2-4 minutes, and inoculated with MRSA for 15 minutes at 37° C. Excess fluid was drained and carriers incubated in BHI overnight followed by quantitative culture.

The results showed that CPC_{sd} inhibited the growth of MRSA on contaminated carriers (CFU count=0). This was compared to control sample that still had a CFU count of 2.54×10⁸. Moreover, pre-coating with CPC_{sd} prevented bacterial contamination of carriers (CFU=0). This was compared to a control with a CFU count of 3.5×10⁸.

A commercial disinfectant containing benzalkonium chloride and ethanol was used as a comparator (Bkc-EtOH), and was identically tested. The comparator also showed similar antibacterial activity.

The effect of a water rinse on sustained disinfectant activity was studied by washing precoated carriers with MILLI-Q (by transferring them into 2 mL Milli Q autoclaved water and removed in 2-3 seconds) followed by exposure to MRSA for 15 minutes and the number of colony forming units (CFUs) were determined after incubation for about 16-24 hours at 37° C. A commercial disinfectant containing benzalkonium chloride and ethanol was used as a comparator (Bkc-EtOH) and were identically tested. Cells with no disinfectant and phosphate-buffered saline treatment were used as controls.

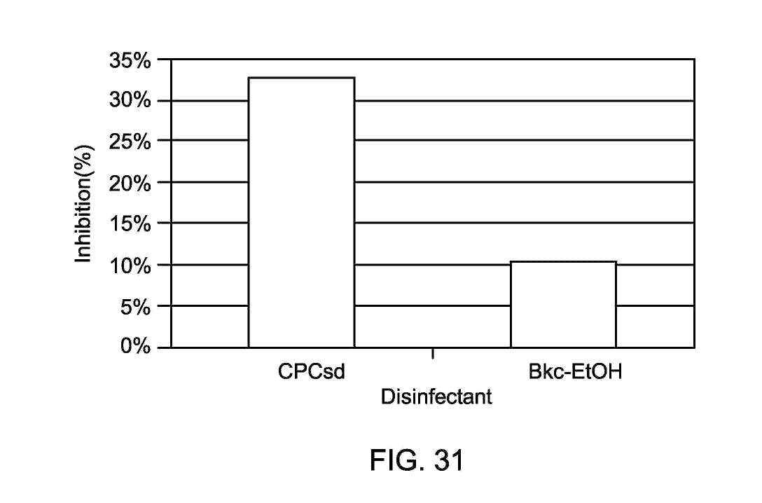

After the water rinse, and after 16-24 hours carriers treated with CPC_{sd} still exhibited 33% reduction in bacterial counts, compared to a 10% reduction in carriers treated with the comparator (FIG. 31). Therefore, the test composition, CPC_{sd}, was able to maintain 3-fold higher activity than the comparator after a water rinse.

### Examples 256

A comparison is presented of the composition tested in Example 255 and common alcohol-based household cleaners.

TABLE XVIII

| | | | | | Anti- | Anti- |
|---|---|---|---|---|---|---|
| | | | | | microbial | microbial |
| | | | | Generally | activity | activity |
| | Flam- | | Irritation | safe for | prior | after |
| | mable | Toxic | Hazard | Children | to rinse | rinse |
| Example 255 | No | No | No | Yes | ~1x | .33x |
| composition | | | | | | |
| Comparative | Yes | Yes | Yes | No | ~1x | .10x |
| Commerical | | | | | | |
| Alcohol-Based | | | | | | |
| Cleaner (58% | | | | | | |
| ethanol) | | | | | | |

While the Example 255 composition is comparable in initial antimicrobial activity it is superior in all safety categories and activity after a water rinse.

### Examples 257-260

Table XIX shows a comparison is made by comparing the composition made in Example 255 (Formulation 2) having no gum and only a low humectant percentage with a composition containing the carbohydrate gum component, such as was described in Example 7 (Formulation 1).

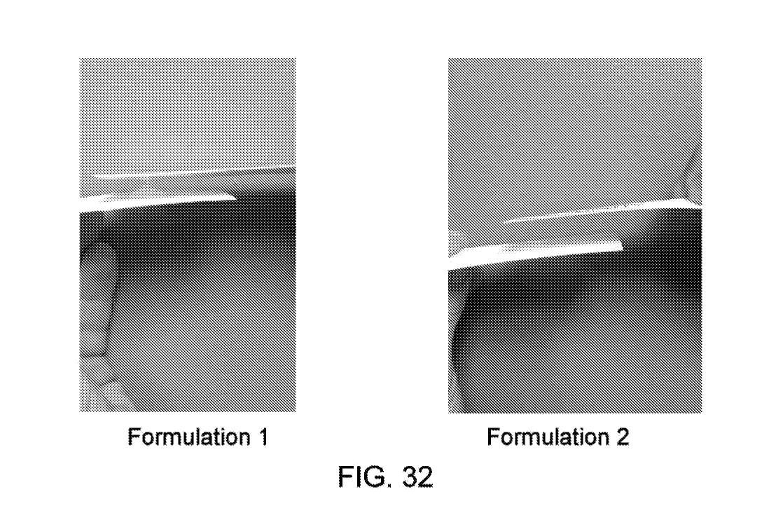

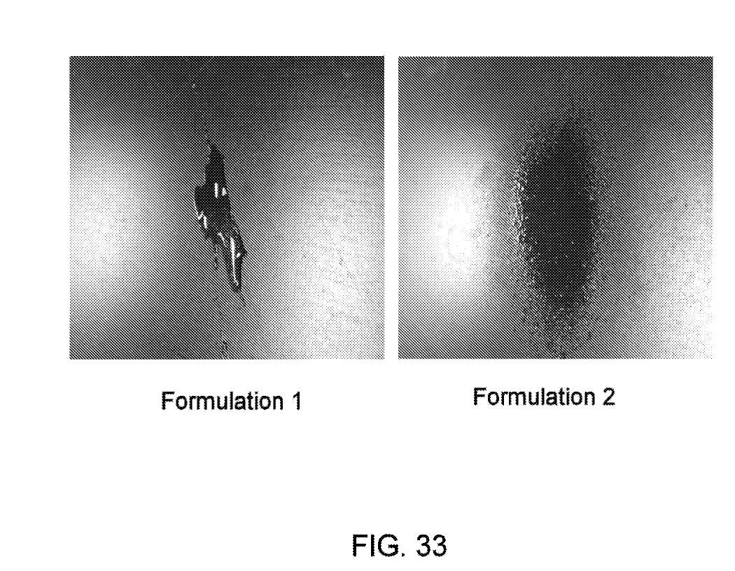

TABLE XIX

| | | | | Example 260 |
|---|---|---|---|---|
| | Example 257 | Example | Example | Surface Area |
| | Stickiness | 258 | 259 | covered per |
| | (Tack) | Viscosity | Thickness | mL |
| Formulation 1 | Higher | Higher | Higher | Lower |
| (Gum) | | | | |
| Formulation 2 | Lower | Lower | Lower | Higher |
| (No gum, low | | | | |
| humectant) | | | | |
| Evidence | FIG. 32 | FIG. 33 | FIG. 32 | FIG. 33 |
| Reference | | and 32 | and 33 | |

In Examples 257-259, shown in FIG. 32, testing was conducted by spraying a 0.25 mL dose of Formulation 1 on one paper-stock card. Then a second paper-stock card was applied for one second and pulled away. The cards were photographed to analyze whether any residue on the original card was continuously attached to both cards after the second card was applied and subsequently removed. As can be seen in FIG. 32, the Formulation 1 composition was still sticking to the test cards at a draw depth of about 0.5 inches. The same method was followed with Formulation 2, and at the same draw there was no residue sticking to the second card. A difference in viscosity and coating thickness can also be seen between the two formulations tested.

In Examples 258-260, shown in FIG. 33, testing for viscosity, layer thickness, and surface area coverage was conducted by spraying a 0.25 mL doses of Formulation 1 and 2 on a lacquer covered fiber board and then photographed. A thicker coating and a smaller surface area was observed in the Formulation 1 test verses the Formulation 2 test. It was also apparent that the viscosity was higher in the Formulation 1 test compared to the Formulation 2 test.

Each of Examples 257-260 were conducted at room temperature, approximately 21° C.

### Example 261

Example 261 was performed to test the antiseptic efficacy of the composition on mammal skin, including human skin, by testing the antibacterial activity of the coating composition in a topical pig skin model with methicillin-resistant Staphylococcus aureus (MRSA) infection. This test was based on the test model reported in Bush et al., “Pig Skin as Test Substitute for Evaluating Topical Antimicrobial Activity,” J. of Clinical Microbiology, p. 343-348 (September 1986).

Harvested pig skin was shaved, cleaned and cut into 3×3 cm squares. Three squares were mounted and inoculated with 0.1 mL of MRSA suspension of 10⁵ cells. After the inoculum dried, one square was treated (sprayed) with about 0.5 mL the coating composition made in Example 255.

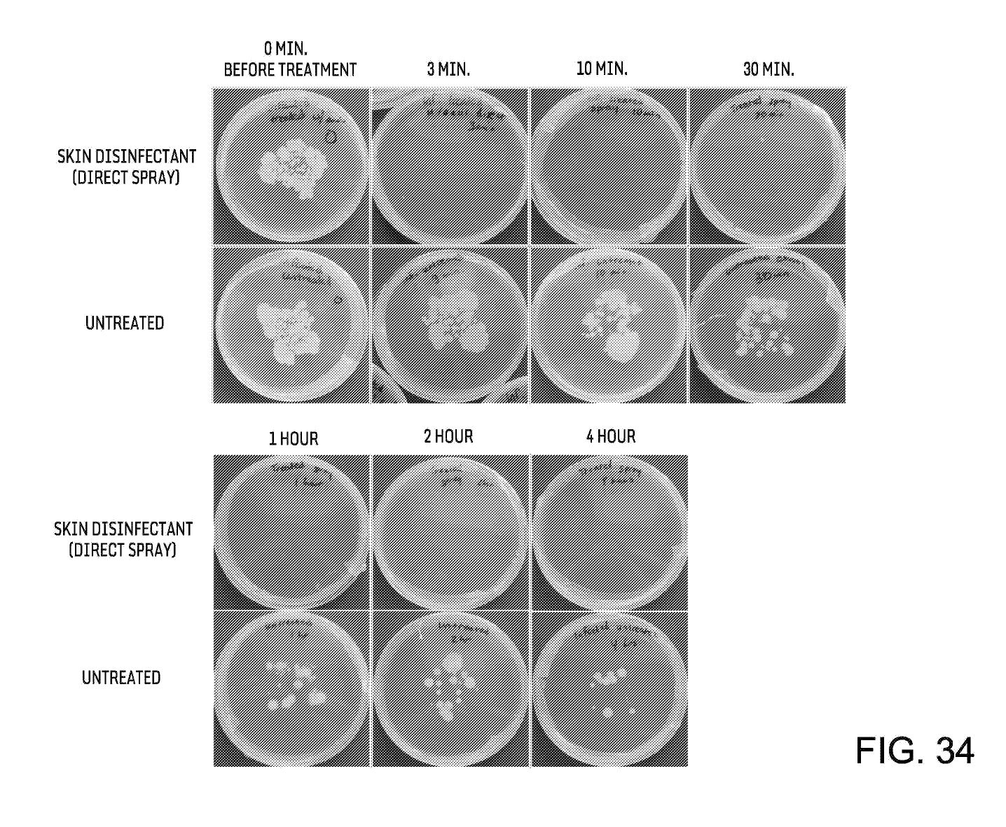

Imprints were made by inverting the mounting holder and pressing the treated and untreated skin onto an agar surface. Imprints were made at 0 minutes prior to treatment 3, 10, and 30 minutes and 1, 2, and 4 hours after the inoculation. FIG. 34 shows the photographs of the agar surfaces after they were incubated for 24 hours at 37° C. at these time intervals.

### Example 262

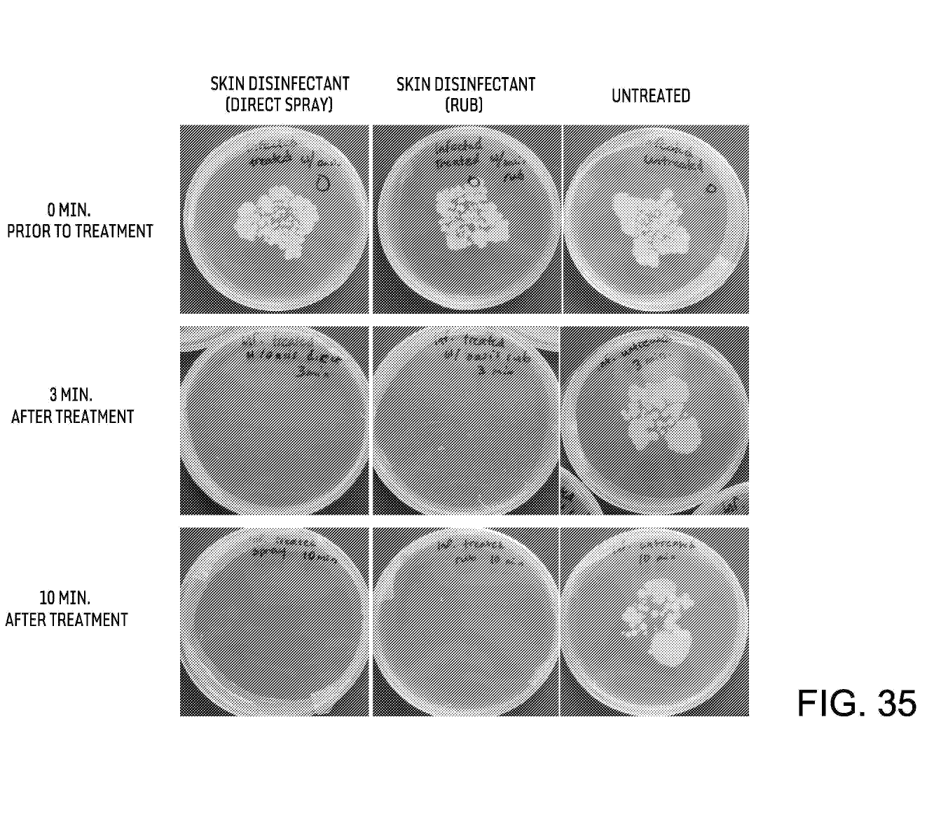

The test of Example 261 was repeated by the same methods, except a spray treated skin sample was rubbed against another inoculated square in order to distribute the compound in a manner simulating hand application. Imprints of an untreated example, the spray treated sample, and the rubbed treated sample were made at 0 minutes prior to treatment, and 3 and 10 minutes after inoculation. FIG. 35 shows the photographs of the agar surfaces after they were incubated for 24 hours at 37° C. at these time intervals. An imprint was also made at 6 hours and showed continued efficacy with no microorganism colonies.

### Example 263

The test of Example 262 was repeated except the pig skin samples were inoculated with Candida instead of MRSA. Imprints were made at the same time intervals as in Example 262 and showed continued efficacy with no colonies formed.

1. A method for treating a body surface with an active agent, the method comprising:
administering a composition in a therapeutically effective amount to a body surface, the composition forming a barrier coating on the body surface that is active to sustain contact with the body surface for a duration of at least about one hour;
the composition meets the following requirements:
about 0.0001%≦C≦about 0.4%;
about 0.07%≦H≦about 70%;
optionally, an antimicrobial A; and
a therapeutically effective amount of X;
or
0%≦C≦about 0.4%;
about 55%≦H≦about 70%;
optionally, an antimicrobial A; and
a therapeutically effective amount of X;
wherein all percentages are by weight of the total composition;
wherein C is a carbohydrate gum, H is a humectant, and X is the active agent, the active agent being active to produce a therapeutic effect on the body.

2. The method of claim 1, wherein the active agent is present in an amount of at least 0.0005% and is less than previously accepted minimum dosages of the active agent.

3. The method of claim 1, wherein the active agent is one or more selected from the group consisting of: antacids, probiotics, vitamins, nutraceuticals, silver, anti-oxidants, cold and flu symptom medicaments, anti-prions, immunostimulators, anti-diarrheals, anti-nausea, topical anesthetics, topical analgesics, anti-pruritics (anti-itch), anti-allergen medicaments, topical steroids, decongestants, cough suppressants, burn or sunburn treatments, expectorants, anti-histamines, topical NSAIDs, topical salicylates, hair-regrowth agents, erectile dysfunction treatment agents, topical antibiotics, topical wound care agents, antioxidant agents, hemorrhoid treatment agents, topical antifungal agents, anti-acne agents, scar treatment agents, psoriasis treatment agents, dry scalp treatment agents, anti-dandruff agents, lice killing agents, hair care agents, body odor amelioration agents, antiperspirants, and foot odor amelioration agents.

4. (canceled)

5. The method of claim 1, wherein the composition comprises an antimicrobial agent, wherein the antimicrobial agent is a monoquaternary ammonium compound or pharmaceutically acceptable salt thereof.

6. The method of claim 1, further comprising:
wherein the step of administering the composition occurs in response to:
a. identifying a contaminated environment that an individual with an elevated risk condition is present in or is going to be present in, wherein the contaminated environment is known or expected to be contaminated with harmful viral, fungal, or bacterial microorganisms; or
b. observing a contamination event in an environment wherein an individual with an elevated risk condition is present in the environment or is going to be present in the environment.

7. (canceled)

8. The method of claim 1, wherein the composition is a solution.

9. The method of claim 1, wherein the therapeutically effective amount of the composition is non-toxic for human consumption.

10. The method of claim 1, wherein the body surface is skin.

11. A method for killing microorganisms on a mammal skin surface comprising:
applying a composition that comprises an antimicrobial agent onto the skin surface;
in the coating, killing or neutralizing microorganisms encountered on the surface and encountered from the environment after the applying step is performed;
forming a coating layer on the skin surface;
the composition comprising a humectant and an antimicrobial agent;
the antimicrobial comprising a monoquaternary ammonium compound or pharmaceutically acceptable salt thereof;
the coating layer having antimicrobial cidal or static activity for at least about one hour.

12. The method of claim 11, wherein the coating composition comprises less than about 50% by weight of humectant, and is essentially free of a carbohydrate gum.

13. The method of claim 11, wherein the coating composition meets the following requirements:
about 0.07%≦H≦about 10%; and
0.0005%<A
wherein all percentages are by weight of the total composition;
wherein H is the humectant and A is the antimicrobial agent.

14. The method of claim 11, wherein the antimicrobial is cetylpyridinium chloride.

15. The method of claim 11, wherein the coating layer has a draw depth of about 0.5 inches or less when applied in a 0.25 mL dosage on a 1 inch diameter area of a paper-stock card, before separating entirely from a second paper-stock card set against the coating layer.

16. The method of claim 11, wherein the microorganisms are selected from the group consisting of: Candida, Pneumonia, MRSA, Streptococcus, P. gingivalis, S. pyogenes, S. pneumonia, S. mutans, S. aureus, Y. enterocolitica, Acinetobacter bumanii, Streptococcus sanguis, S. oxalis, S. mitis, S. salivarius, S. gordonii, Aggregatibacter actinomycetemcomitans, Fusobacterium nucleatum, C. albicans, C. krusei, C. tropicalis, C. glabrata, HIV, EBV, influenza viruses, microorganisms that cause upper respiratory infections, Centipeda periodontii, Eikenella corrodens, Enterobacteriaceae, Fusobacterium nucleatum subsp. nucleatum, Fusobacterium nucleatum subsp. polymorphum, Fusobacterium nucleatum subsp. vincentii, Fusobacterium periodonticum, Porphyromonas endodontalis, Prevotella (Bacteroides) melaninogenica, Prevotella intermedia, Bacteroides (Bacteroides) loescheii, Solobacterium moorei, Tannerella forsythia (Bacteroides forsythus), Treponema denticola, Clostridium difficile, Bordetella pertussis, Burkholderia, Aspergillus fumigatus, Penicillium spp, Cladosporium, HPV, cold, Klebsiella pneumoniae, Salmonella choleraesuis, Escherichia coli (0157:H7), Trichophyton mentagrophytes, Rhinovirus Type 39, Respiratory Syncytial Virus, Poliovirus Type 1, Rotavirus Wa, Influenza A Virus, Herpes Simplex Virus Types 1 & 2, and Hepatitis A Virus.

17. (canceled)

18. The method of claim 11, wherein the skin surface is the hands, and the composition is applied at least daily.

19. A composition for body surface treatment, the composition comprising:
about 0.0001%≦C≦about 0.4%;
about 0.07%≦H≦about 70%;
optionally an antimicrobial A; and
a therapeutically effective amount of X; or
0%≦C≦about 0.4%;
about 55%≦H≦about 70%;
optionally an antimicrobial A; and
a therapeutically effective amount of X;
wherein all percentages are by weight of the total composition;
wherein C is a carbohydrate gum, H is a humectant, A is an antimicrobial, and X is an active agent;
the active agent being selected from the group consisting of one or more of:
antacids, probiotics, vitamins, nutraceuticals, silver, anti-oxidants, cold and flu symptom medicaments, anti-prions, immunostimulators, anti-diarrheals, anti-nausea, topical anesthetics, topical analgesics, anti-pruritics (anti-itch), anti-allergen medicaments, topical steroids, decongestants, cough suppressants, burn or sunburn treatments, expectorants, anti-histamines, topical NSAIDs, topical salicylates, hair-regrowth agents, erectile dysfunction treatment agents, topical antibiotics, topical wound care agents, antioxidant agents, hemorrhoid treatment agents, topical antifungal agents, anti-acne agents, sear treatment agents, psoriasis treatment agents, dry scalp treatment agents, anti-dandruff agents, lice killing agents, hair care agents, body odor amelioration agents, antiperspirants, and foot odor amelioration agents.

20. The composition of claim 19, wherein the antimicrobial comprises a monoquaternary ammonium compound or pharmaceutically acceptable salt thereof.

21. The composition of claim 19, wherein the active agent is a body amelioration agent, an antiperspirant, or both.

22. The composition of claim 19, wherein the composition is a solution and upon placing on a 140 U.S. mesh 95 to 100% by weight of the composition passes through after 30 seconds.

23. The composition of claim 19, wherein the active agent is eucalyptol.
